(19) United States
(12) Patent Application Publication
(10) Pub. No.: US 2011/0054332 A1
(43) Pub. Date: Mar. 3, 2011

(54) Blood Pressure Measuring Cuff

(76) Inventors: Leonid Shturman, Nyon (CH); Lela Nadirashvili, Nyon (CH)

(21) Appl. No.: 12/744,068

(22) PCT Filed: Nov. 21, 2008
(86) PCT No.: PCT/EP08/65994

§ 371 Date: Sep. 28, 2010

(30) Foreign Application Priority Data
Nov. 23, 2007 (GB) 0722998.2

(51) Int. Cl. A61B 5/02; A61B 17/135

(52) U.S. Cl. 600/499; 606/202

(57) ABSTRACT

An inflatable cuff comprising an ak inflatable bladder and a strap member which forms a sleeve configured to extend around an arm of a person is disclosed. The sleeve shaped strap member has a waist portion which carries the bladder and a pair of longitudinally extending edges. The longitudinally extending edges of the waist portion are attached to complimentary interlocking halves of a zip fastener to enable the longitudinally extending edges to be zipped together to reduce the circumference of the waist portion of the sleeve and secure the cuff in position around a person's arm whose blood pressure is to be measured.

The present invention relates to an inflatable cuff used for taking human blood pressure measurements, and more particularly, to a cuff that is placed around the arm of a patient either by the patient themselves or by others.

The most common method for non-invasive arterial blood pressure measurement uses an inflatable cuff which is wrapped around an arm above the elbow. The cuff is temporarily inflated to a pressure which cuts off blood flow in the brachial artery. As the air is controllably released, blood pressure is estimated by detecting “Korotkoff” sounds using either a stethoscope placed below the cuff or a microphone built-in the cuff. The stethoscope or the microphone is positioned over the artery. The sounds named after physician Korotkoff are produced by disturbances in the flow of blood through a partially occluded brachial artery. The brachial artery is occluded by the externally applied cuff pressure. As the cuff pressure is reduced the systolic pressure equals the cuff pressure at the time the first Korotkoff sound is detected. The diastolic pressure is identified when silence occurs. An aneroid pressure gauge is commonly used as a pressure indicating device. Devices of this type are commonly referred to as “sphygmomanometers.” Blood pressure cuffs are well known in the prior art and comprise an elongated, flexible strap member (band) having a predetermined length and width. Each cuff usually has a pair of walls which are joined together around their peripheries to form a compartment (pouch) into which an inflatable rubber bladder is inserted. A hose portion coupled to the bladder extends out from the compartment through an opening in the periphery. The strap member is adapted to be wrapped around the upper arm. Attachment means such as Velcro brand Hook and Loop fabric fastener is provided with the strap member to hold the strap member on the arm.

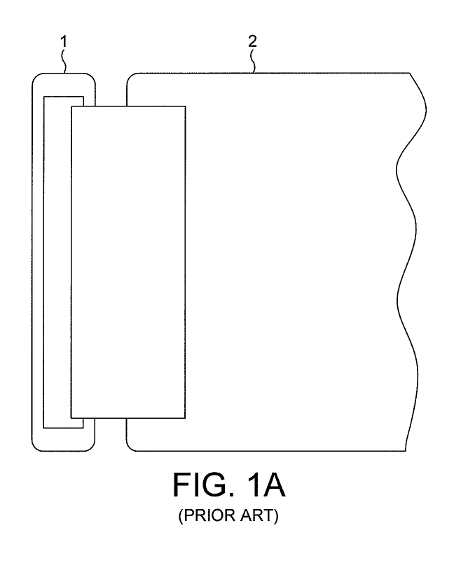

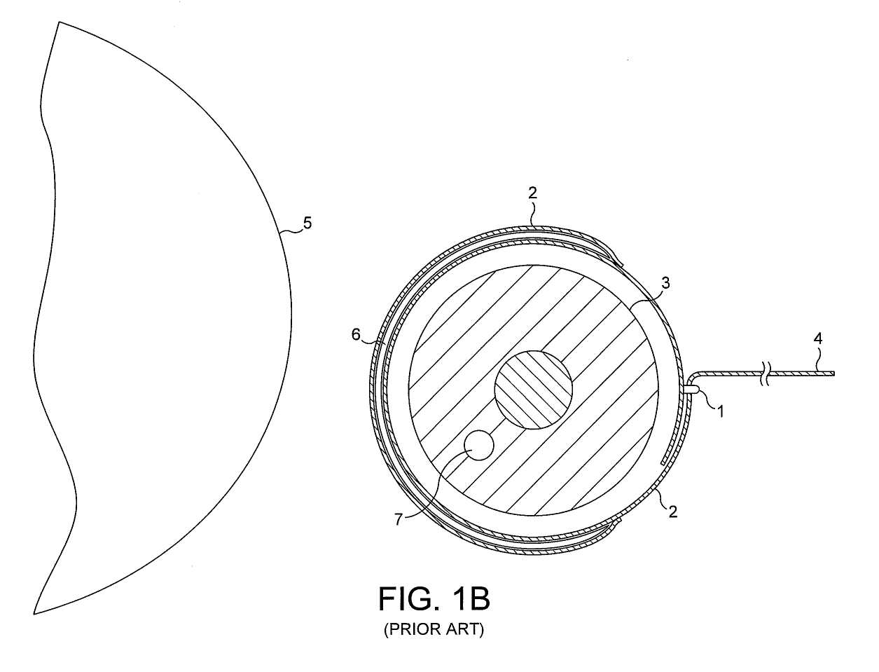

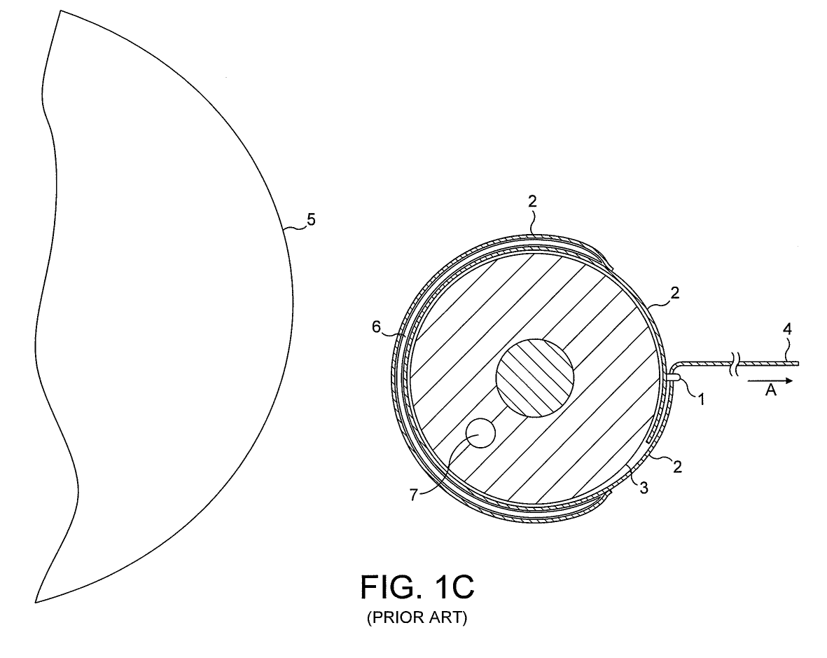

FIG. 1A shows a self-applied blood pressure cuff of the prior art which includes a buckle in the form of an elongated cinch ring 1 to which one end of the strap member 2 is attached and through which the other, or free, end of the strap member is passed to form a sleeve. U.S. Pat. No. 4,832,040 to Gray E. Ruff and U.S. Pat. No 5,271,409 to Jack M. Millay provide examples of design and method of use of blood pressure cuffs with above described metal cinch rings. Application of the cuff around an arm is started with the insertion of the free end of the strap member through the buckle of the strap member to form a sleeve into which the arm is inserted. It should be noted that the cuff with the cinch ring has both the “loop” and the “hook” sides of the Velcro brand Hook and Loop fabric fastener located on the same side of the strap member, and both the “loop” and the “hook” sides should face out when the sleeve is formed. FIG. 1B shows that the cinch ring 1 of the cuff should be positioned along an outer side of the upper arm 3. FIG. 1C illustrates that application of the cuff around the upper arm 3 is continued by pulling the free end 4 of the strap member 2 away from the body trunk 5 to tighten the cuff around the arm. The direction of the force which pulls the free end 4 of the strap member 2 away from the body trunk 5 is indicated by an arrow A. FIG. 1D illustrates that application of the cuff around the upper arm 3 is finished by pulling the strap member 2 over the buckle 1 toward the body trunk 5 to press together portions of the strap member with complementary sides of the Velcro brand Hook and Loop fabric fastener without loosening the strap member. The direction of the force which pulls the strap member 2 over the buckle 1 toward the body trunk 5 is indicated by an arrow B.

When wrapping the blood pressure cuff around the upper arm, it is desirable for accuracy of measurement that the center of the inflatable bladder 6 be located over the brachial artery 7 on the inner side of the arm. It should be understood that pulling the free end of the strap member towards the body trunk over the buckle located on the outer side of the arm inevitably results in pulling the strap member around the arm. FIG. 1E illustrates that pulling the strap member 2 around the arm may cause rotation of the cuff around the arm 3 and movement of the inflatable bladder 6 away from its proper position over the brachial artery 7. The present invention seeks to provide an inflatable blood pressure cuff which can be tightened around the upper arm without any risk of inadvertently rotating it around the arm. The present invention also seeks to provide an inflatable blood pressure cuff which, even prior to inflation, has a consistently tight fit around the arm when used repetitively by the same patient over a long period of time such as weeks, months or even years. This is particularly important for patients who are treated for their hypertension with potent pharmaceuticals such as beta-blockers. The present invention also seeks to provide an inflatable blood pressure cuff which is continuous with a sleeve of an upper body garment and can be closed and opened around the arm with ease by the patient themselves.

According to one aspect of the invention, there is provided an inflatable cuff comprising an air inflatable bladder and a strap member which forms a sleeve configured to extend around an arm of a person, the sleeve shaped strap member having a waist portion which carries the bladder and has a pair of longitudinally extending edges, characterised in that the longitudinally extending edges of the waist portion are attached to complimentary interlocking halves of a zip fastener to enable the longitudinally extending edges to be zipped together to reduce the circumference of the waist portion of the sleeve and secure the cuff in position around a person's arm whose blood pressure is to be measured.

Preferably, the bladder extends around the arm under an outer wall of the sleeve shaped strap member, the waist portion being closeable around the arm prior to inflation of the bladder which, after inflation, compresses the arm and stops the flow of blood through the brachial artery of the arm, the waist portion having a circumferential length extending in a direction around the arm and a width extending in a direction along the length of the arm, the waist portion having a pair of waist tightening edges which span the width of the waist portion and which, when the waist portion is open, are spaced away from each other by the circumferential length of the waist portion, the waist tightening edges of the waist portion forming segments of elongated waist tightening edges of the sleeve shaped strap member.

In one embodiment, the waist tightening edges of the waist portion extend beyond the width of the waist portion to form auxiliary segments of the elongated waist tightening edges of the strap member, the auxiliary segments being attached to the interlocking halves of the zip fastener which also extends beyond the waist portion and which is configured for fastening together all segments of the elongated waist tightening edges of the sleeve shaped strap member.

The inflatable cuff of the invention may comprise a single zip fastener which fastens together the waist tightening edges.

Preferably, the strap member has the waist portion that carries the bladder and an auxiliary portion which extends along the length of the arm away from the waist portion, both the waist portion and the auxiliary portion having waist tightening edges which form respective segments of the elongated waist tightening edges of the strap member, said waist tightening edges of both the waist portion and the auxiliary portion of the strap member being attached to the complementary halves of the zip fastener, so that the waist portion and the auxiliary portion of the strap member can be closed and opened by the same zip fastener.

In one embodiment, the strap member has the waist portion that carries the bladder and an auxiliary portion which extends along the length of the arm away from the waist portion, the waist portion and the auxiliary portion each having a pair of waist tightening edges which they share with the elongated waist tightening edges of the strap member and which are attached to the complementary halves of the zip fastener, so that the waist portion and the auxiliary portion of the strap member can be closed and opened by the same zip fastener.

The auxiliary portion of the strap member may have a central segment and a peripheral segment, the central segment is located next to the waist portion of the strap member and can be closed and opened by the zip fastener while the peripheral segment is spaced away from the waist portion of the strap member and is undividable by the zip fastener, the zip fastener extending through the waist portion of the strap member and the central segment of the auxiliary portion and having a slider located such that its movement opens the waist portion of the strap member before the central segment of the auxiliary portion and closes the central segment of the auxiliary portion before the waist portion of the strap member.

In one preferred embodiment, the auxiliary portion of the strap member extends up along the arm and is configured for being pulled up along the arm when a slider of the zip fastener is moved down along the arm so that the strap member is not moved beyond its appropriate position along the length of the arm, at least one of the slider and the auxiliary portion of the strap member being manipulated by someone other than the person who's blood pressure is measured.

The auxiliary portion of the strap member may extend down along the arm and be configured for being pulled down along the arm when a slider of the zip fastener is moved up along the arm so that the strap member is not moved beyond its appropriate position along the length of the arm, at least one of the slider and the auxiliary portion of the strap member being manipulated by someone other than the person who's blood pressure is measured.

The auxiliary portion of the strap member may extend distally towards the hand and is configured for forming a loop around the hand, said loop anchoring the strap member to the hand and preventing movement of the strap member beyond its appropriate position along the length of the arm when a slider of the zip fastener is moved up along the arm by the person themselves.

In one embodiment, the auxiliary portion of the strap member extends along an outer surface of the forearm and its anchoring loop extends across the palm.

In one embodiment, the auxiliary portion of the strap member extends distally towards the hand and is configured for forming a loop around the thumb, said loop anchoring the strap member to the thumb and preventing movement of the strap member beyond its appropriate position along the length of the arm when a slider of the zip fastener is moved up along the arm by the person themselves.

In one embodiment, the auxiliary portion of the strap member extends distally towards the hand and is configured for forming a loop(s) around the finger(s), said loop(s) anchoring the strap member to the finger(s) and preventing movement of the strap member beyond its appropriate position along the length of the arm when a slider of the zip fastener is moved up along the arm by the person themselves.

In one embodiment, the auxiliary portion of the strap member extends distally towards the hand and is configured for being grasped by the hand prior to using the opposite arm for moving the slider of the zip fastener up along the arm by the person themselves.

In a preferred embodiment, the auxiliary portion of the strap member extends along an outer surface of the forearm.

Preferably, the auxiliary portion of the strap member is configured for extending along an outer surface of the forearm and has a finger hole(s) near its distal end, said finger hole(s) configured for anchoring the auxiliary portion of the strap member to the finger(s) and preventing movement of the strap member beyond its appropriate position along the length of the arm when a slider of the zip fastener is moved up along the arm by the person themselves.

The auxiliary portion of the strap member may have an adjustable length. For example, the auxiliary portion of the strap member comprises two parts which are detachably fastened to each other by a Velcro brand Hook and Loop fabric fastener to enable the length of the auxiliary portion to be adjusted. Alternatively, the auxiliary portion of the strap member comprises two parts which are detachably button fastened to each other, one part having a button and the other a plurality of buttonholes spaced away from each other along the length of the other part, to enable the length of the auxiliary portion to be adjusted. Alternatively, the auxiliary portion of the strap member comprises two parts which are detachably button fastened to each other, one part having a buttonhole and the other a plurality of buttons spaced away from each other along the length of the other part, to enable the length of the auxiliary portion to be adjusted.

In one embodiment, the strap member has the waist portion that carries the bladder and a pair of auxiliary portions which extend away from the waist portion in opposite directions along the length of the arm, one of the two auxiliary portions being a distal auxiliary portion and extending towards the hand and the other being a proximal auxiliary portion and extending towards the shoulder, the waist portion and the auxiliary portions each having a pair of waist tightening edges which form respective segments of the elongated waist tightening edges of the strap member, said waist tightening edges of the waist portion and of both the distal and proximal auxiliary portions of the strap member being attached to the complementary halves of the zip fastener, so that the waist portion and the auxiliary portions of the strap member can be closed and opened by the same zip fastener.

In a preferred embodiment, the strap member has the waist portion that carries the bladder and a pair of auxiliary portions which extend away from the waist portion in opposite directions along the length of the arm, one of the two auxiliary portions being a distal auxiliary portion and extending towards the hand and the other being a proximal auxiliary portion and extending towards the shoulder, the waist portion and the auxiliary portions each having a pair of waist tightening edges which they share with the elongated waist tightening edges of the strap member and which are attached to the complementary halves of the zip fastener, so that the waist portion and the auxiliary portions of the strap member can be closed and opened by the same zip fastener.

The distal and proximal auxiliary portions of the strap member may have central segments and peripheral segments, the central segments are located next to the waist portion of the strap member and can be closed and opened by the zip fastener while the peripheral segments are spaced away from the waist portion of the strap member and are undividable by the zip fastener, the zip fastener extending through the waist portion and the central segments of the auxiliary portions and having a slider located such that opening and closing of the strap member is started with one of the two central segments and finished with the other.

Each of the auxiliary portions of the strap member can be configured for being pulled along the arm in a direction which is opposite to the direction in which the slider of the zip fastener is moved along the arm, so that movement of the strap member beyond its appropriate position around the arm can be prevented, at least one of the slider and the auxiliary portion of the strap member being manipulated by someone other than the person whose blood pressure is measured.

The proximal auxiliary portion of the strap member may be configured for being pulled up along the arm when the slider of the zip fastener is moved down along the arm so that the strap member is not moved beyond its appropriate position around the arm, at least one of the slider and the proximal auxiliary portion of the strap member being manipulated by someone other than the person whose blood pressure is measured.

The distal auxiliary portion of the strap member may be configured for being pulled down along the arm when the slider of the zip fastener is moved up along the arm so that the strap member is not moved beyond its appropriate position around the arm, at least one of the slider and the distal auxiliary portion of the strap member being manipulated by someone other than the person whose blood pressure is measured.

In one embodiment, the strap member has the waist portion that carries the bladder and an auxiliary portion which extends along the length of the arm away from the waist portion and is continuous with a sleeve of an upper body garment, both the waist portion and the auxiliary portion each having a pair of waist tightening edges which they share with the elongated waist tightening edges of the strap member and which are attached to the complementary halves of the zip fastener, so that the waist portion and the auxiliary portion of the strap member can be closed and opened by the same zip fastener.

In one embodiment, the strap member has the waist portion that carries the bladder and an auxiliary portion which extends along the length of the arm away from the waist portion, both the waist portion and the auxiliary portion of the strap member being continuous with a sleeve of an upper body garment, the waist portion and the auxiliary portion of the strap member having waist tightening edges which they share with the elongated waist tightening edges of the strap member and which are attached to the complementary halves of the zip fastener, so that the waist portion and the auxiliary portion of the strap member can be closed and opened by the same zip fastener.

In another embodiment, the strap member has the waist portion that carries the bladder and a pair of auxiliary portions which extend away from the waist portion in opposite directions along the length of the arm and are comprised from a sleeve of an upper body garment, a portion of the sleeve of the garment which extends from the waist portion up along the arm comprising a proximal auxiliary portion, and a portion of the sleeve of the garment which extends from the waist portion down along the arm comprising a distal auxiliary portion, each of the auxiliary portions having a pair of waist tightening edges which extend up and down the sleeve of the garment from the waist portion of the strap member to form auxiliary waist tightening edges of the strap member, the auxiliary waist tightening edges being attached to the complementary interlocking halves of the strap member's single zip fastener, the strap member's single zip fastener extending through the waist portion of the strap member and continuing to extend up and down the sleeve of the garment away from the waist portion of the garment and having a slider located such that opening and closing of the strap member is started with one of two auxiliary portions of the strap member and finished with the other.

The strap member may have a waist portion that carries the bladder and a pair of auxiliary portions which extend away from the waist portion in opposite directions along the length of the arm and which are continuous with a sleeve of an upper body garment, one of two auxiliary portions being a distal auxiliary portion and extending towards the hand and the other being a proximal auxiliary portion and extending towards the shoulder, the waist portion and each of the auxiliary portions having a pair of waist tightening edges which they share with the elongated waist tightening edges of the strap member and which are attached to the complementary halves of the strap member's single zip fastener, so that the waist portion and the auxiliary portions of the strap member can be closed and opened by the same zip fasten.

The zip fastener may extend up the sleeve of the garment but terminate short of the shoulder area of the garment.

In one embodiment, the proximal auxiliary portion of the strap member is continuous with sleeve and shoulder areas of the upper body garment, the zip fastener extending into and terminating within the shoulder area of the garment.

The proximal auxiliary portion of the strap member may be continuous with sleeve and shoulder areas of the upper body garment, the zip fastener extending into the shoulder area of the garment but terminating short of the collar of the garment.

In one embodiment, the proximal auxiliary portion of the strap member is continuous with sleeve and shoulder areas of the upper body garment, the zip fastener extending into the shoulder area of the garment but terminating short of a neck margin of the garment.

The distal auxiliary portion of the strap member may extend towards the wrist, the waist tightening edges of the distal auxiliary portion of the strap member extending down into the forearm area of the sleeve of the garment, so that the sleeve of the garment located down from the waist portion of the strap member can be opened and closed around the arm and forearm by the strap member's single zip fastener.

The distal auxiliary portion of the strap member can be continuous with a long sleeve of the garment, the zip fastener extending down into the forearm portion of the sleeve but terminating short of a distal end of the sleeve of the garment, the distal end of the sleeve having a cuff which extends around the wrist and is configured for anchoring the sleeve to the wrist, said anchoring of the sleeve to the wrist restricting movement of the strap member up along the arm beyond its appropriate position around the arm when the opposite arm is used to move the slider of the zip fastener up along the arm by the person themselves.

A portion of the sleeve which is continuous with the distal auxiliary portion of the strap member may have a strap extension which is configured for anchoring the sleeve to the hand, said anchoring of the sleeve to the hand of the arm restricting movement of the strap member up along the arm beyond its appropriate position around the arm when the opposite arm is used to move the slider of the zip fastener up along the arm by the person themselves.

The strap member can be continuous with one of two sleeves of the upper body garment and extending around the arm which is used for measuring the blood pressure, the other sleeve extending around the opposite arm and having a cuff which extends around the wrist and is configured for anchoring the sleeve to the wrist, said anchoring of the other sleeve to the wrist of the opposite arm restricting movement of the strap member down along the arm beyond its appropriate position around the arm when the opposite arm is used to move the slider of the zip fastener down along the arm by the person themselves.

In one embodiment, the strap member is continuous with one of two sleeves of the upper body garment, the sleeve continuous with the strap member having a first strap extension which is configured for anchoring the sleeve to the hand and restricting movement of the strap member up along the arm when the opposite arm is used to move the slider of the zip fastener up along the arm, the other sleeve extending around the opposite arm and having a second strap extension which is configured for anchoring the other sleeve to the hand of the opposite arm and restricting movement of the strap member down along the arm when the opposite arm is used to move the slider of the zip fastener down along the arm.

In one embodiment, the strap member is continuous with one of two sleeves of the upper body garment and extends around the arm which is used for measuring the blood pressure, the other sleeve extending around the opposite arm and having a strap extension which is configured for anchoring the other sleeve to the hand of the opposite arm, said anchoring of the other sleeve to the hand of the opposite arm restricting movement of the strap member down along the arm beyond its appropriate position around the arm when the opposite arm is used to move the slider of the zip fastener down along the arm by the person themselves.

The strap extension may be configured for being grasped by the hand of the opposite arm prior to using the opposite arm and its strap extension holding hand for moving the slider of the zip fastener down along the arm by the person themselves.

The strap extension can be configured for being passed along an outer surface of the hand first, moved under the thumb and across an inner surface of the hand, and finally grasped using fingers prior to using the opposite arm and its strap extension holding hand for moving the slider of the zip fastener down along the arm by the person themselves.

In one embodiment, the strap extension is configured for passing along an outer surface of the hand and forming an anchoring loop around the hand, said anchoring loop extending across an inner surface of the hand distal to the base of the thumb but proximal to bases of the fingers.

The strap extension can be configured for passing along an outer surface of the hand and forming an anchoring loop(s) around the finger(s).

In one embodiment, the strap extension is configured for passing along an outer surface of the hand and has a finger hole(s) near its distal end, said finger hole(s) configured for anchoring the strap extension to the finger(s).

In an alternative embodiment, the strap member includes a non-stretchable bridging element which, after closing of the zip fastener, is extended over the zip fastener to interconnect regions of the strap member which adjoin each of the two halves of the zip fastener, the bridging element interconnects the regions of the strap member which are located on both sides of the closed zip fastener and prevents the interlocked halves of the zip fastener from separating from each other by reducing the strain applied to the zip fastener when the inflatable bladder is inflated.

The strap member may include a stretchable strip which extends along one of the two halves of the zip fastener and a non-stretchable bridging element which, after closing the zip fastener, is extended over the stretchable strip and the zip fastener to interconnect regions of the strap member which are spaced away from each other by the stretchable strip and the closed zip fastener, the stretchable strip and the non-stretchable bridging element preventing the interlocked halves of the zip fastener from separating from each other by reducing the strain applied to the zip fastener when the inflatable bladder is inflated, the non-stretchable bridging element configured to prevent the interlocked halves of the zip fastener from separating even when air pressure in the fully inflated bladder has reached about 300 mm mercury, while the stretchable strip configured to stretch and reduce to a limited extent an initial strain on the zip fastener during an early stage of the inflation when air pressure in the bladder only started to rise.

In one embodiment, the stretchable strip of the strap member is configured to stretch only until the strain becomes applied to the non-stretchable bridging element which, when strained, prevents further stretching of the stretchable strip.

The non-stretchable bridging element may be configured for detachable fastening to the regions of the strap member located along-side both halves of the zip fastener by a Velcro brand Hook and Loop fabric fastener, a “hook” side of the fastener being carried by each of the two regions of the strap member and a “loop” side by the bridging element.

The non-stretchable bridging element can be configured for detachable fastening to the regions of the strap member located along-side both halves of the zip fastener by a Velcro brand Hook and Loop fabric fastener, a “loop” side of the fastener being carried by each of the two regions of the strap member and a “hook” side by the bridging element.

In one embodiment, the non-stretchable bridging element is configured for detachable fastening by a Velcro brand Hook and Loop fabric fastener to the regions of the strap member which are spaced away from each other by the zip fastener and the stretchable strip, one of the regions located along-side one half of the zip fastener and the other along-side the stretchable strip, a “hook” side of the fastener being carried by each of the two regions of the strap member and a “loop” side by the bridging element.

Preferably, the non-stretchable bridging element is configured for detachable fastening by a Velcro brand Hook and Loop fabric fastener to the regions of the strap member which are spaced away from each other by the zip fastener and the stretchable strip, one of the regions located along-side one half of the zip fastener and the other along-side the stretchable strip, a “loop” side of the fastener being carried by each of the two regions of the strap member and a “hook” side by the bridging element.

In one embodiment, the bridging element is permanently attached to the wall of the strap member near either end of the zip fastener.

The bridging element can be permanently fastened to the region of the strap member located along-side one half of the zip fastener and is configured for detachable fastening by a Velcro brand Hook and Loop fabric fastener to the region of the strap member located along-side the other half of the zip fastener.

The bridging element may be permanently fastened to the region of the strap member located along-side the stretchable strip and is configured for detachable fastening by a Velcro brand Hook and Loop fabric fastener to the region of the strap member located along-side one half of the zip fastener.

The bridging element may be permanently fastened to the region of the strap member located along-side one half of the zip fastener and is configured for detachable fastening by a Velcro brand Hook and Loop fabric fastener to the region of the strap member located along-side the stretchable strip.

According to one embodiment of the invention, the waist portion of the strap member has inner and outer walls and carries the bladder within a pouch formed between said walls of the waist portion of the strap member, both the pouch and the bladder extending around the arm.

The waist portion of the strap member may have a pair of longitudinally extending ends, one end coinciding with one of the two waist tightening edges of the waist portion and the other end being spaced away from the other waist tightening edge so that parts of the wall of the waist portion located close to the longitudinally extending ends overlap each other, when the waist portion is closed by the zip fastener.

In one embodiment, the auxiliary portion(s) of the strap member can be formed from the zip fastener alone.

In another embodiment, the auxiliary portion(s) of the strap member can be formed only from the zip fastener and from the sleeve of the upper body garment.

The waist portion of the strap member can be configured such that, when the zip fastener is open, the edges of the sleeve are separated from each other to enable a person to insert their arm into the sleeve from one end of the sleeve shaped strap member.

Reference is made to an “appropriate” position of the blood pressure cuff or its strap member around the upper arm For the avoidance of doubt, and for the purpose of this specification, the cuff is positioned appropriately when the waist portion of the strap member is not encroaching on either the armpit or the elbow. It should be noted that commercially available blood pressure cuffs have average width of about 12 centimetres and are positioned about 2 centimetres above the elbow. Reference is also made to a “longitudinally” extending end(s) or edge(s) of the strap member. For the avoidance of doubt, and for the purpose of this specification, the longitudinally extending end or edge of the strap member is the end or edge which extends along the length of the upper arm.

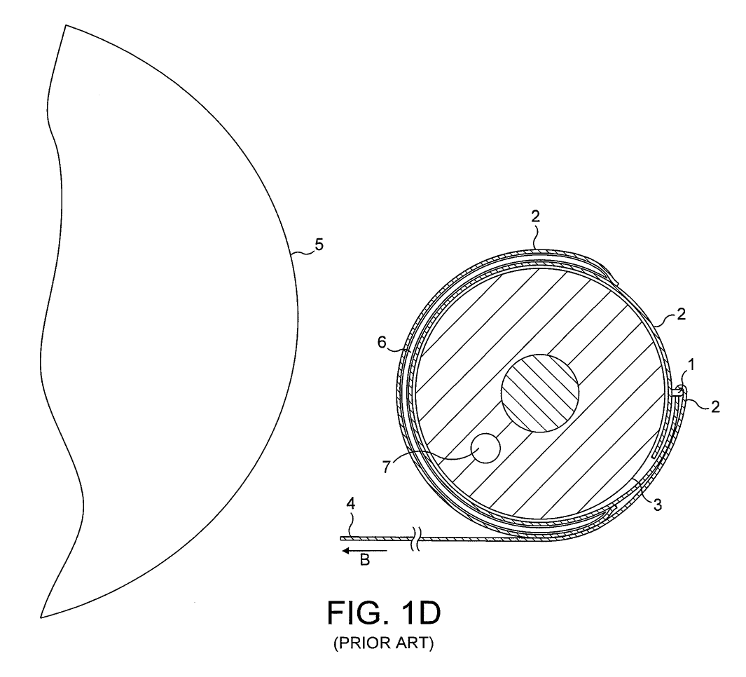

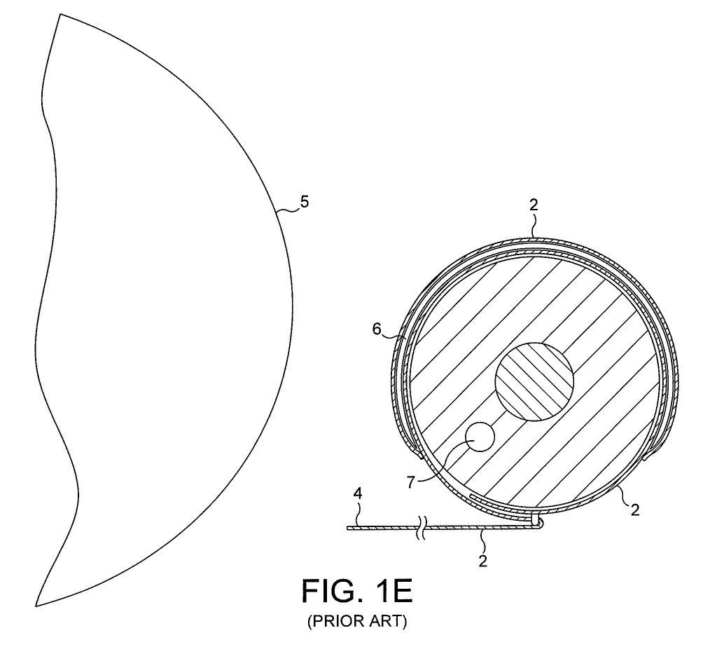

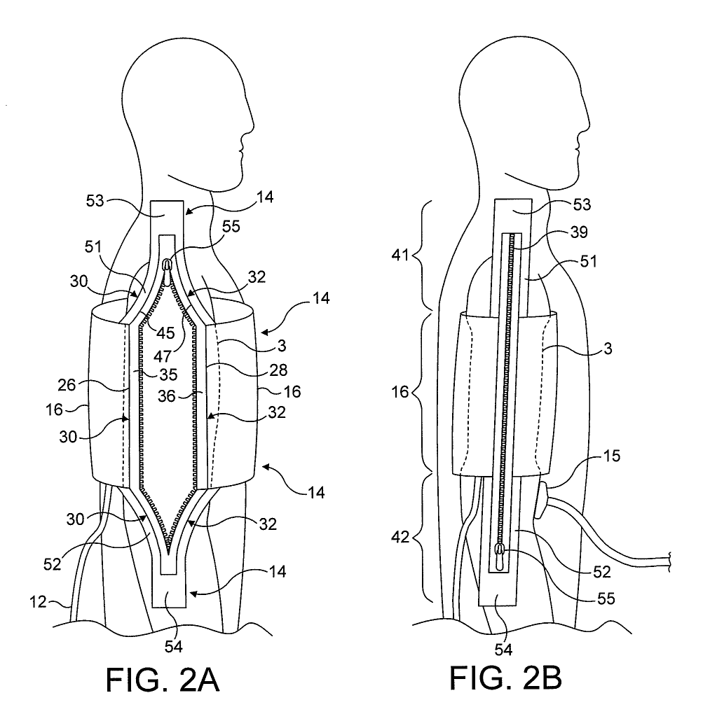

Embodiments of the invention will now be described, by way of example only, with reference to FIGS. 2A to 12C of the accompanying drawings, in which:

FIG. 1A shows a self-applied blood pressure cuff of the prior art which includes a buckle in the form of an elongated cinch ring to which one end of a strap member is attached and through which the other, or free, end of the strap member is passed to form a sleeve;

FIG. 1B shows that the cinch ring of the cuff should be positioned along an outer side of the upper arm;

FIG. 1C illustrates that application of the cuff around the upper arm is continued by pulling the free end of the strap member away from the body trunk to tighten the cuff around the arm;

FIG. 1D illustrates that application of the cuff around the upper arm is finished by pulling the strap member over the buckle toward the body trunk to press together portions of the strap member with complementary sides of the Velcro brand Hook and Loop fabric fastener;

FIG. 1E illustrates that pulling the strap member around the arm may cause rotation of the cuff around the arm and movement of the inflatable bladder away from its proper position over the brachial artery;

FIG. 2A illustrates an inflatable blood pressure cuff of the invention. The cuff is shown attached around the upper arm of the patient. The cuff includes a sleeve shaped strap member which carries an air inflatable bladder. A zip fastener is provided to fasten together elongated waist tightening edges of the strap member. The zip fastener is shown in an open position;

FIG. 2B shows the cuff of FIG. 2A, wherein the zip fastener has been closed to decrease the circumference of the cuff around the person's arm;

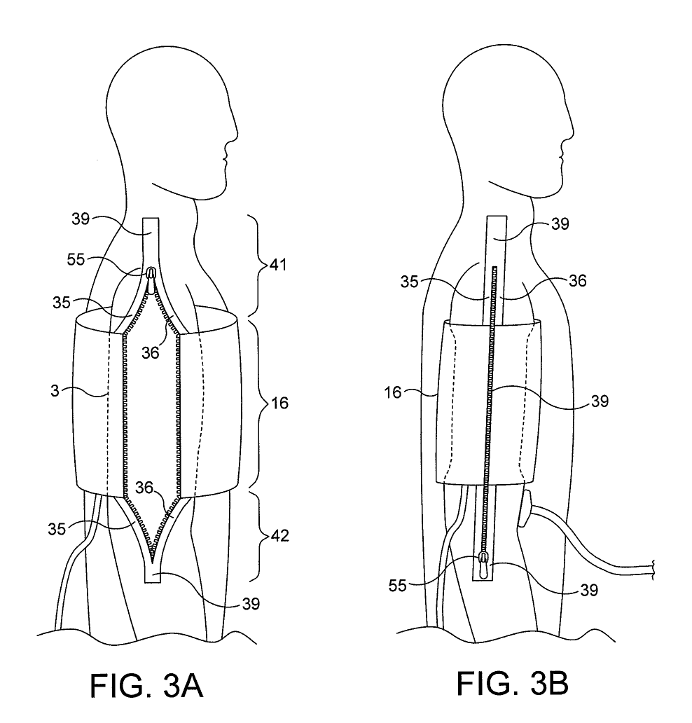

FIGS. 3A and 3B show that auxiliary portions of the strap member can be formed from the zip fastener alone;

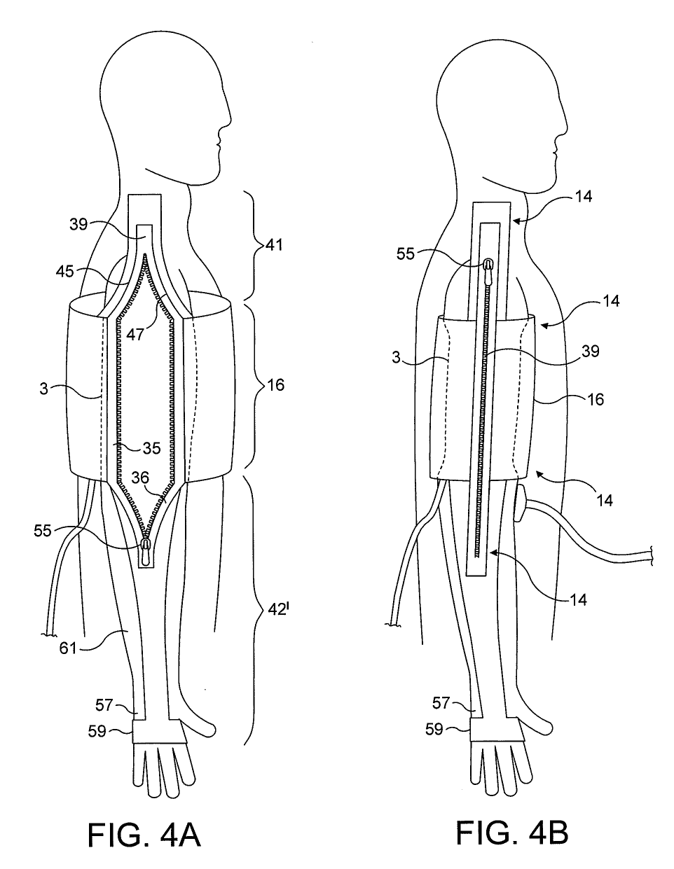

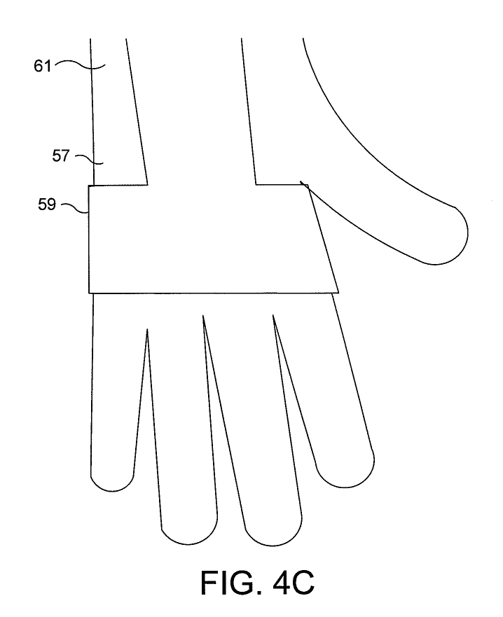

FIG. 4A shows an alternative embodiment of a cuff of the invention in an open position, which is the same as the cuff shown in FIGS. 2A and 2B, except that the cuff includes a longitudinal extension for anchoring the cuff to a person's hand to prevent movement of the cuff in a proximal direction along the person's upper arm when the zip fastener is being closed;

FIG. 4B shows the cuff of FIG. 4A with the zip fastener closed to reduce the circumference of the cuff around the person's arm;

FIG. 4C shows a close-up of the longitudinal extension of FIGS. 4A and 4B;

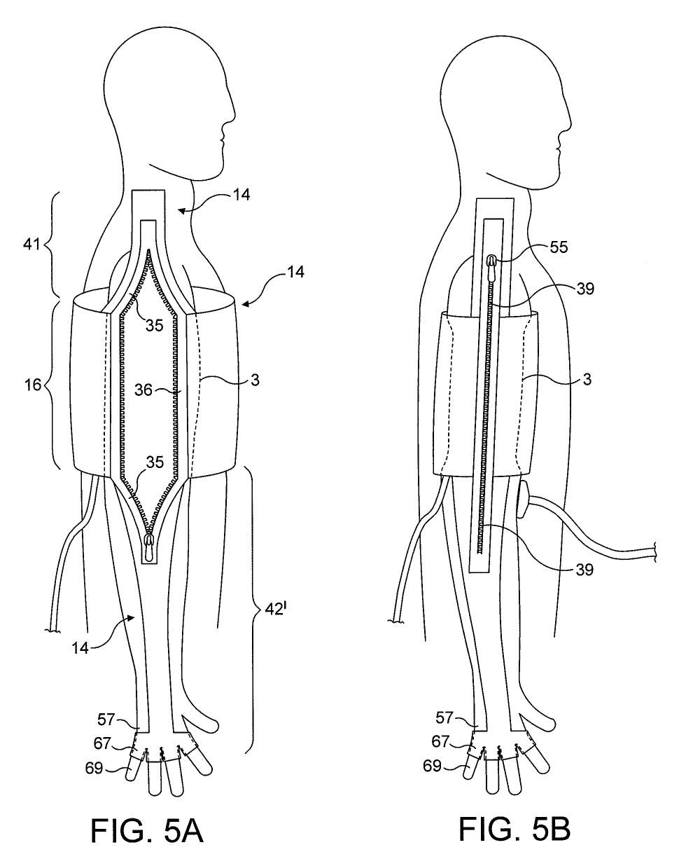

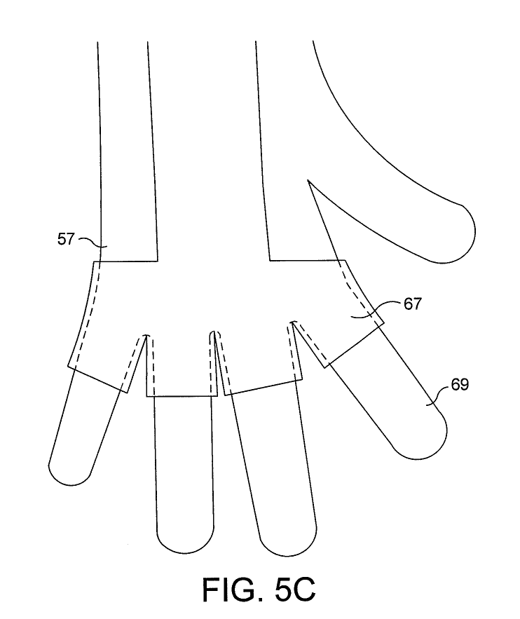

FIG. 5A shows a further alternative embodiment of a cuff of the invention in an open position, which is the same as the cuff shown in FIGS. 4A and 4B, except that the cuff includes a modified longitudinal extension configured for anchoring the cuff to a person's hand at the base of the fingers;

FIG. 5B shows the cuff of FIG. 5A with the zip fastener closed to reduce the circumference of the cuff around the person's arm;

FIG. 5C shows a close-up of the longitudinal extension of FIGS. 5A and 5B;

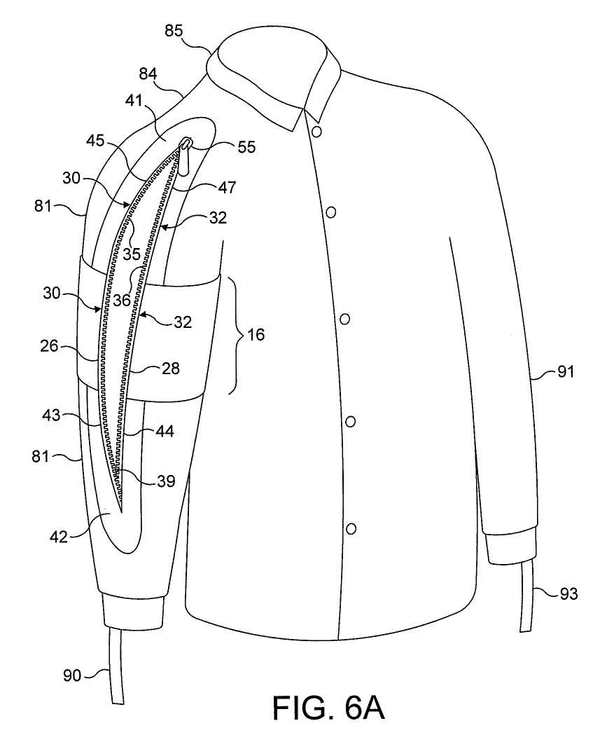

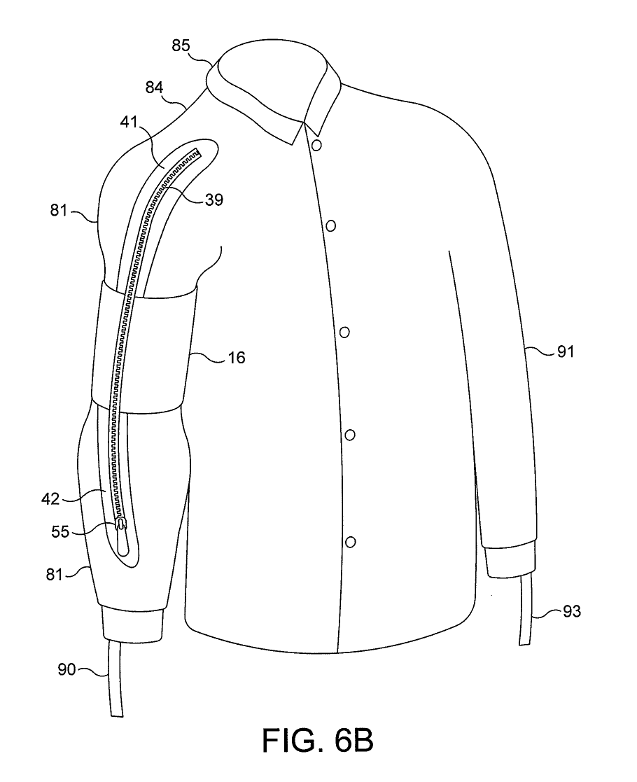

FIGS. 6A and 6B show a modified embodiment of the invention in which the cuff is incorporated into the sleeve of an upper body garment;

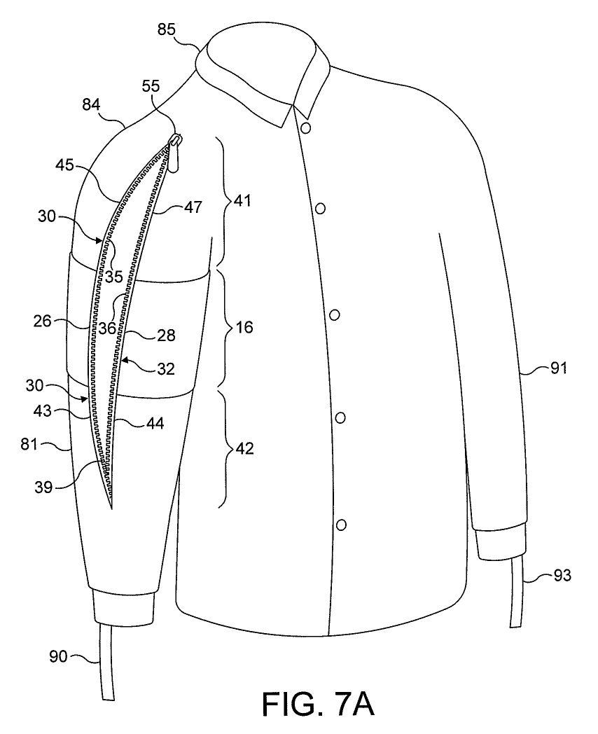

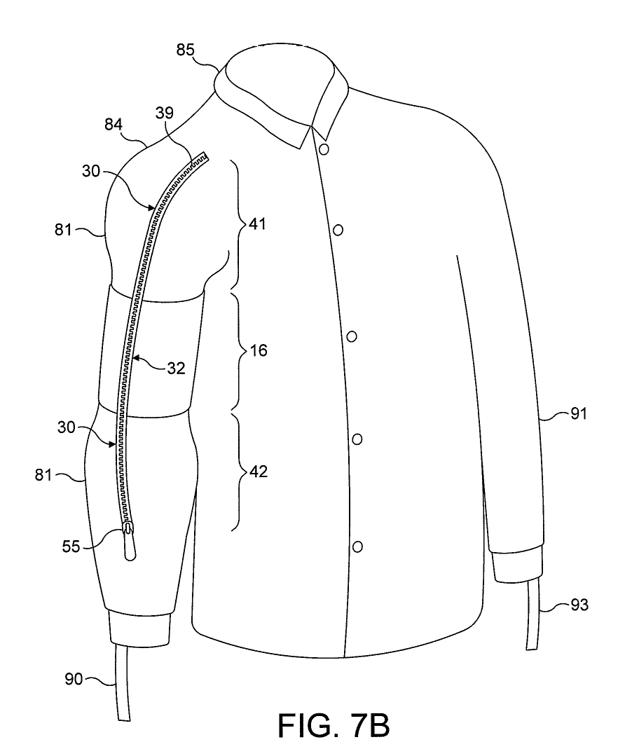

FIGS. 7A and 7B show that the auxiliary portions of the strap member can be formed from only the sleeve of the garment and the zip fastener extending through the sleeve;

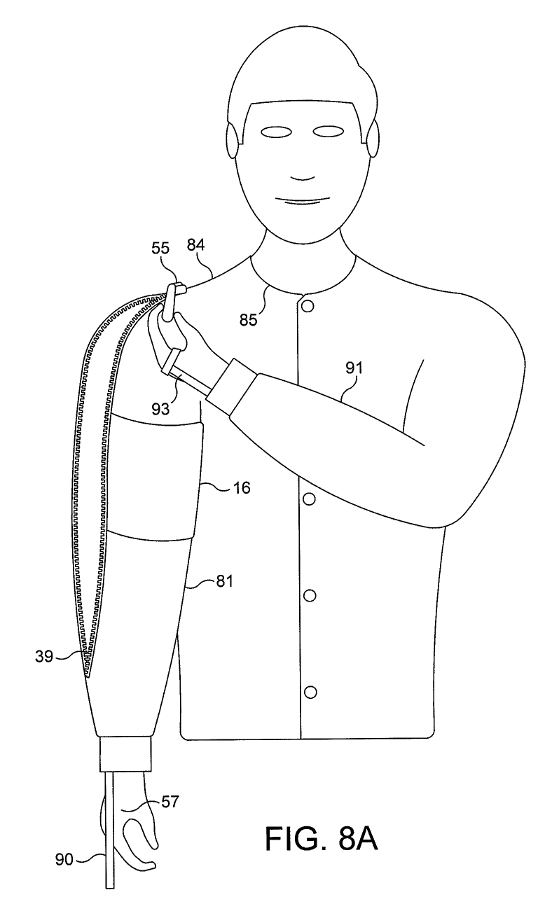

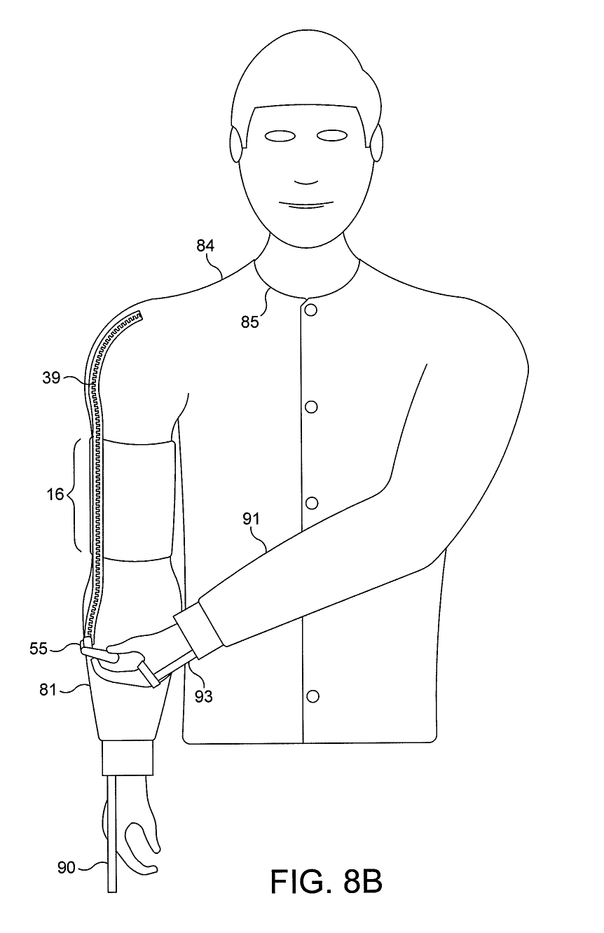

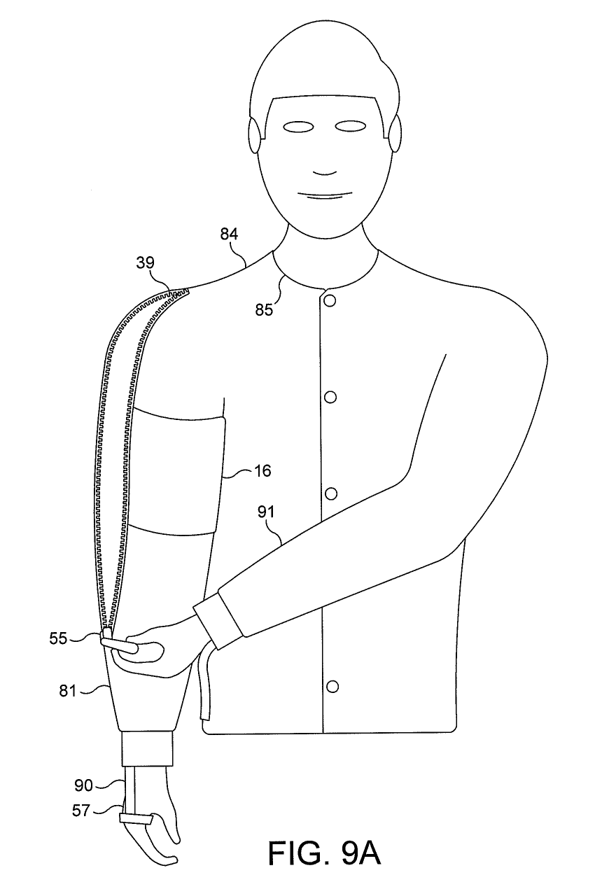

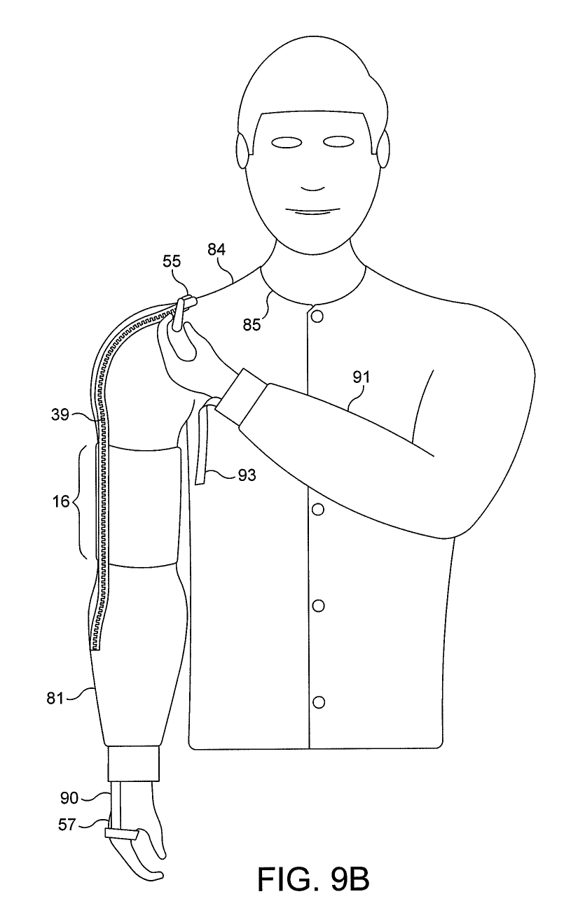

FIGS. 8A through 9B illustrate how a person may use the cuff of the invention shown in FIGS. 6A through 7B;

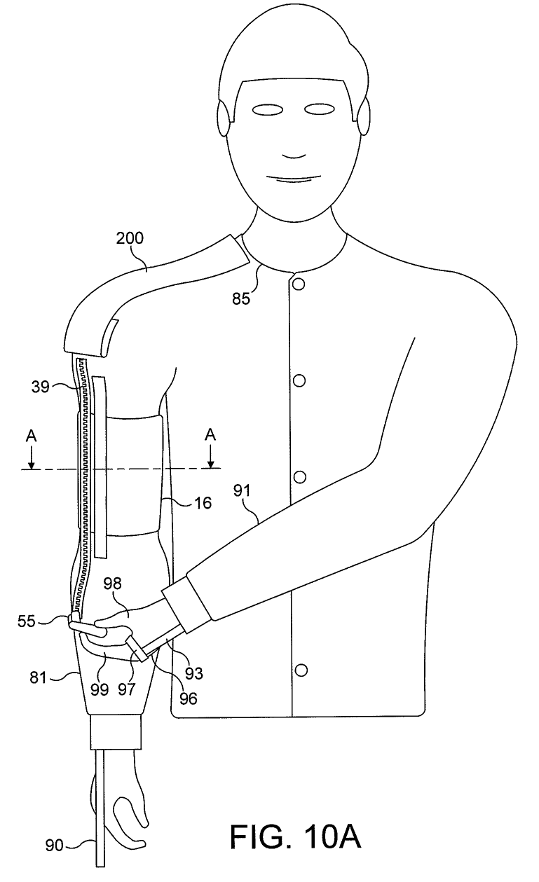

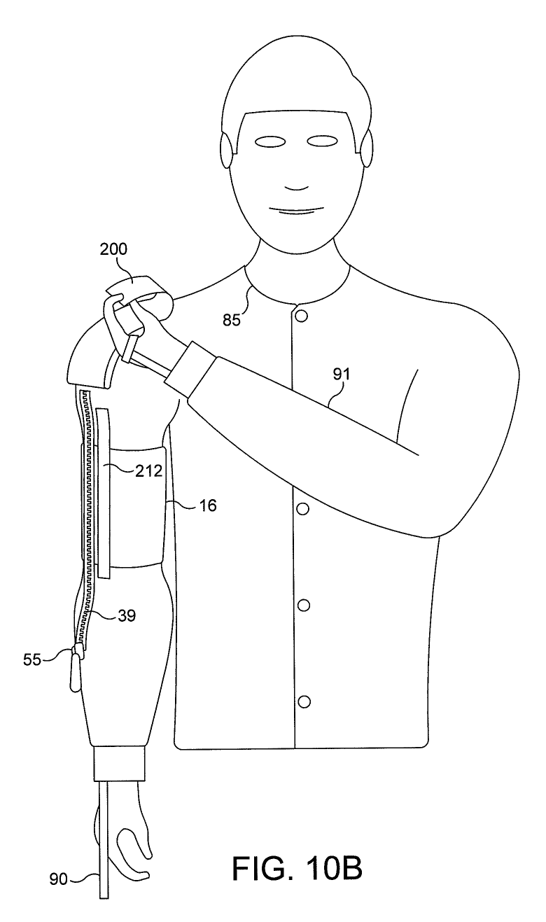

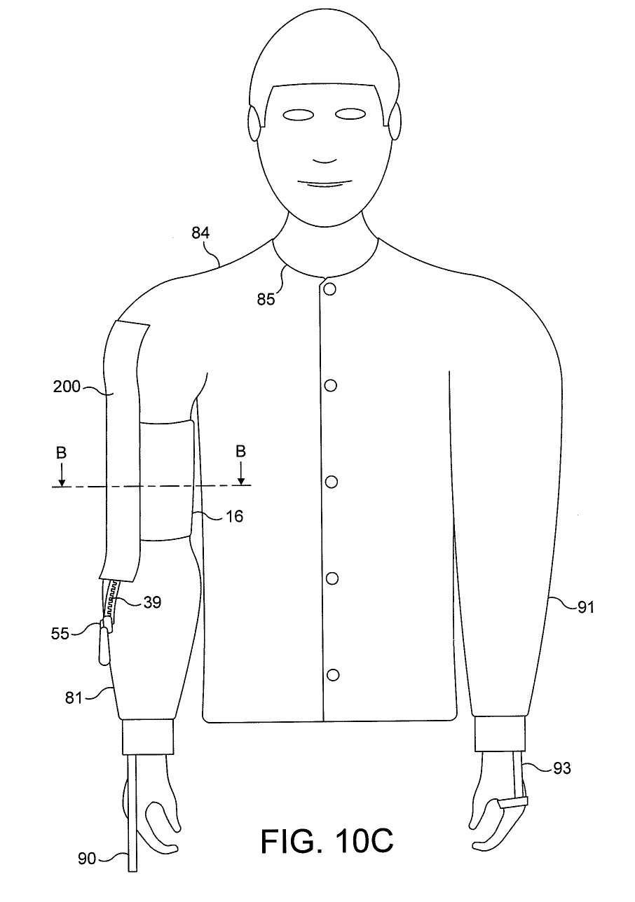

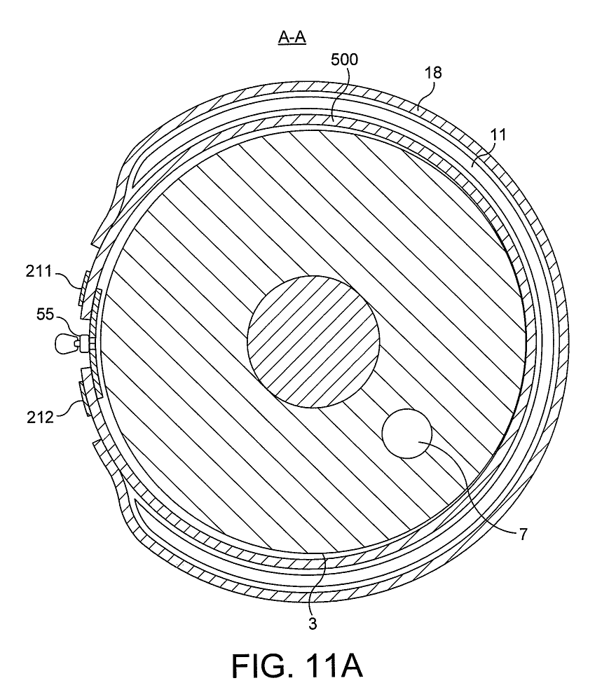

FIGS. 10A through 10C show how a non-stretchable bridging element may be extended over the zip fastener and may interconnect with portions of the strap member located along both sides of the zip fastener;

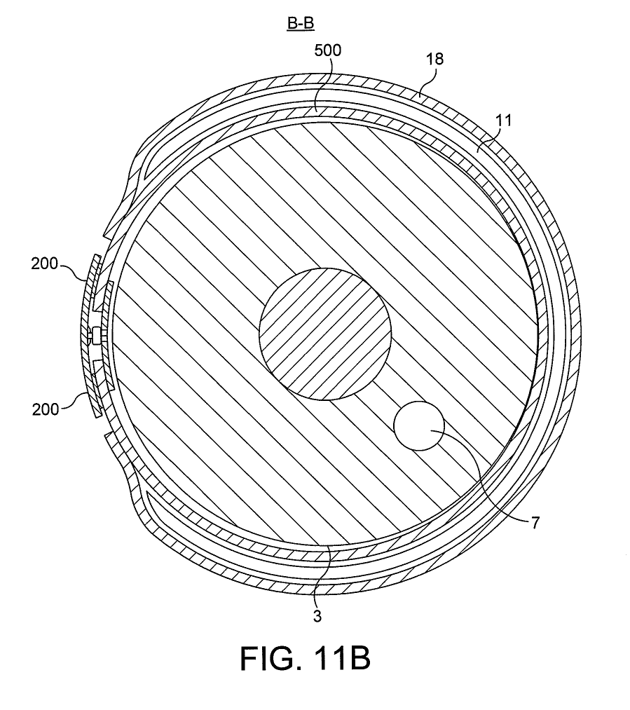

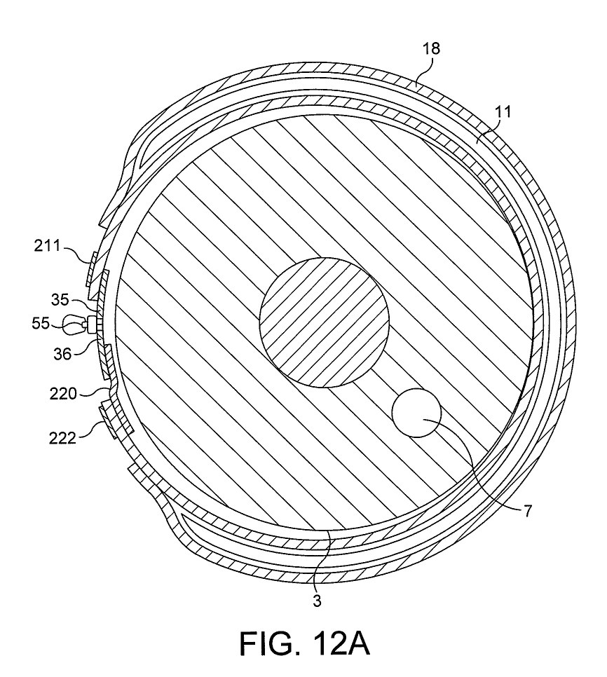

FIGS. 11A and 11B are transverse cross sections of FIGS. 10A and 10C, respectively; and

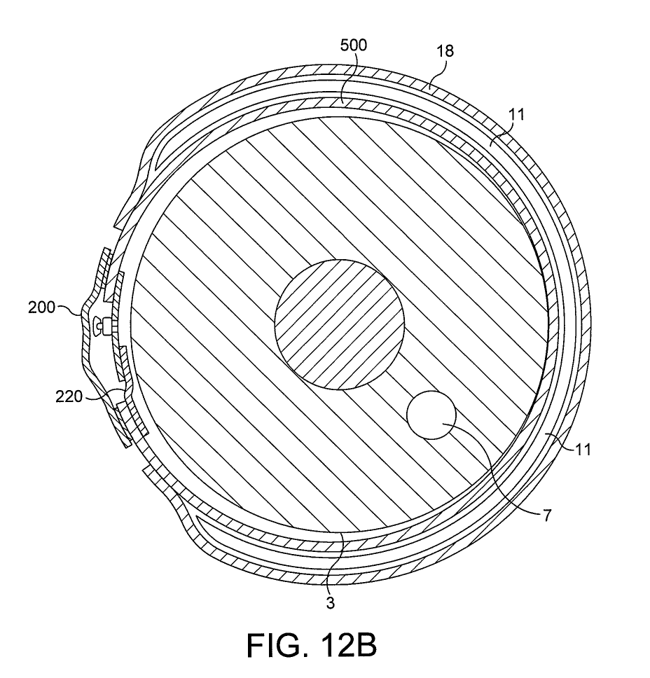

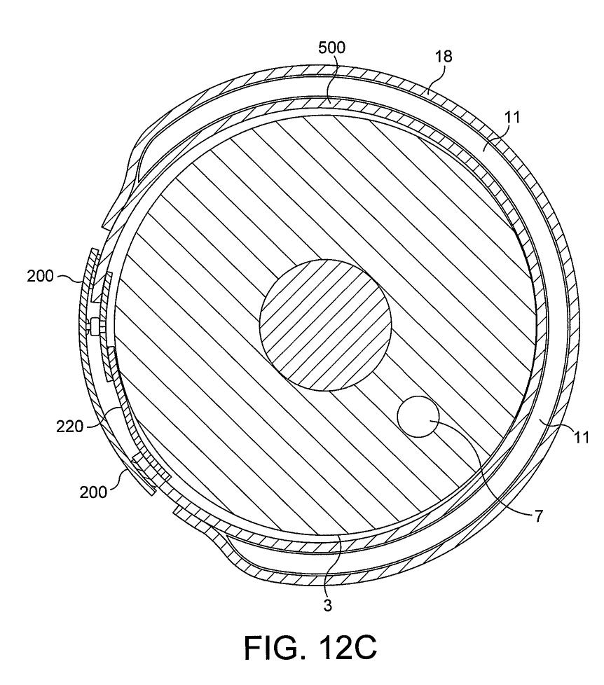

FIGS. 12A and 12C illustrate a modified embodiment which incorporates the non-stretchable bridging element and a stretchable strip which together prevents the interlocked halves of the zip fastener from separating from each other by reducing the strain applied to the zip fastener when the inflatable bladder is inflated.

Referring now to the drawings, FIGS. 2A through FIG. 5C illustrate an inflatable cuff for measuring human blood pressure in accordance with the first embodiment of the cuff of the invention. The cuff is shown attached around the upper arm of the patient. The cuff is comprised of an air inflatable bladder and a strap member 14 which carries the bladder. The air inflatable bladder 11 is shown in FIGS. 11A through 12C. The strap member 14 forms a sleeve around an arm 3 of the patient. FIGS. 11A through 12C illustrate that the bladder 11 extends around the arm 3 of the patient under an outer wall 18 of the sleeve shaped strap member 14. The wall 18 limits the length of an outer circumference of an inflated bladder 11 when the waist portion 16 is closed around the arm 3 prior to inflation of the bladder 11. The bladder 11, when inflated, compresses the arm 3 and stops the flow of blood in the brachial artery of the arm. The waist portion 16 has a circumferential length and a width. The waist portion 16 extends around the arm 3 along its circumferential length and encircles the arm when the waist portion 16 is closed. The width of the waist portion 16 is measured along the length of the arm 3 and in the preferred embodiment of the invention it depends on the circumferential length of the waist portion 16. It has been determined that a width of the inflatable bladder 11 should be approximately 40% of the circumference of the arm to properly occlude the brachial artery. Therefore the increase in the circumferential length of the waist portion 16 should be associated with the increase of its width.

The waist portion has a pair of longitudinally extending waist tightening edges 26, 28 which are attached to complimentary interlocking halves 35,36 of a zip fastener 39 to enable the longitudinally extending edges 26,28 of the waist portion 16 to be zipped together to reduce the circumference of the waist portion 16 of the sleeve shaped strap member 14 and secure the cuff in position around a person's arm whose blood pressure is to be measured. The waist tightening edges 26,28 span the width of the waist portion 16 of the sleeve shaped strap member 14. The waist portion 16 is closeable around the arm 3 before inflation of the bladder 11 which, after inflation, compresses the arm 3 and stops flow of blood through the brachial artery 7.

The waist tightening edges 26,28 of the waist portion 16 extend beyond the width of the waist portion 16 to form auxiliary segments of the elongated waist tightening edges 30,32 of the strap member 14. The auxiliary segments may comprise proximal auxiliary segments 45,47 and/or distal auxiliary segments 43,44. The waist tightening edges 26,28 of the waist portion 16 and the auxiliary segments 45,47; 43,44 of corresponding proximal and distal auxiliary portions of the strap member 14 are attached to the interlocking halves 35,36 of the zip fastener 39. The zip fastener 39 also extends beyond the waist portion 16 and is configured for fastening together all segments of the elongated waist tightening edges 30,32 of the sleeve shaped strap member 14.

The elongated waist tightening edges 30, 32 of the strap member 14 are attached to complementary interlocking halves 35, 36 of a zip fastener 39 which is configured for closing the sleeve shaped strap member 14 around the arm 3 by fastening together the interlocking halves of the zip fastener and for opening the sleeve shaped strap member 14 by separating the complementary halves of the zip fastener 39.

According to a preferred embodiment of the invention the zip fastener 39 which closes and opens the strap member 14 around the arm is comprised of a single zip fastener 39.

A conduit 12 is provided to supply pressurised gas from a pressurised gas source (not shown) to inflate the cuff. FIG. 2B shows the cuff of FIG. 2A, wherein the zip fastener 39 has been closed, prior to inflation of the bladder 11, to decrease the circumference of the waist portion 16 of the strap member 14 around the person's arm. The bladder 11 is then inflated. The outer circumference of the bladder 11 is limited by the outer wall 18 of the waist portion 16 and the inflated bladder 11 compresses the arm 3 and stops flow of blood through the brachial artery. A stethoscope 15 is usually used to hear appearance and disappearance of the sounds which correspond to systolic and diastolic pressures and registered by observing indications on a manometer (not shown).

According to a preferred embodiment of the invention the strap member 14 has the waist portion 16 that carries the bladder 11 and at least one auxiliary portion (e. g. 41 or 42) which extends along the length of the arm away from the waist portion 16. FIGS. 2A, 2B show that the waist portion 16 has waist tightening edges 26, 28 and the auxiliary portion 41 has waist tightening edges 45, 47 which form respective segments of the elongated waist tightening edges 30, 32 of the strap member 14. It should be understood that both the waist portion 16 and the auxiliary portion 41 share their respective waist tightening edges 26, 28 and 45, 47 with the elongated waist tightening edges 30, 32 of the strap member 14. The waist tightening edges of both the waist portion 16 and the auxiliary portion 41 of the strap member are attached to the complementary halves 35, 36 of the zip fastener 39, so that the waist portion 16 and the auxiliary portion 41 of the strap member 14 can be closed and opened by the same zip fastener 39.

FIGS. 2A, 2B show that the auxiliary portion 41 of the strap member has a central segment 51 and a peripheral segment 53. The central segment 51 is located next to the waist portion 16 of the strap member and can be closed and opened by the zip fastener 39 while the peripheral segment 53 is spaced away from the waist portion 16 of the strap member and is undividable by the zip fastener 39. The zip fastener 39 extends through the waist portion 16 of the strap member 14 and the central segment 51 of the auxiliary portion 41 and has a slider 55 located such that its movement opens the waist portion 16 of the strap member before the central segment 51 of the auxiliary portion and closes the central segment 51 of the auxiliary portion before the waist portion 16 of the strap member.

FIGS. 2A, 2B show that the auxiliary portion 41 of the strap member 14 extends up along the arm. The auxiliary portion 41 is configured for being pulled up along the arm 3 when a slider 55 of the zip fastener 39 is moved down along the arm so that the strap member 14 is not moved beyond its appropriate position along the length of the arm 3, at least one of the slider 55 and the auxiliary portion 41 of the strap member being manipulated by someone other than the person who's blood pressure is measured.

FIGS. 2A, 2B show that the auxiliary portion 42 of the strap member 14 extends down along the arm. The auxiliary portion 42 is configured for being pulled down along the arm 3 when a slider 55 of the zip fastener 39 is moved up along the arm so that the strap member 14 is not moved beyond its appropriate position along the length of the arm 3, at least one of the slider 55 and the auxiliary portion 42 of the strap member being manipulated by someone other than the person who's blood pressure is measured.

FIGS. 3A and 3B show that auxiliary portions 41, 42 of the strap member 14 may be formed from the zip fastener alone. FIGS. 4A and 4B show that the auxiliary portion 42′ of the strap member 14 extends distally towards the hand 57 and is configured for forming a loop 59 around the hand, said loop anchoring the strap member to the hand 57 and preventing movement of the strap member beyond its appropriate position along the length of the arm 3 when a slider 55 of the zip fastener 39 is moved up along the arm by the person themselves to close the zip fastener.

FIGS. 4A, 4B and 4C show the auxiliary portion 42′ of the strap member 14 extends along an outer surface of the forearm and its anchoring loop 59 is configured for extending across the palm.

FIGS. 5A, 5B and 5C show a modification of the auxiliary portion 42′ of the strap member which extends distally towards the hand 57. The modified auxiliary portion 42′ of FIGS. 5A, 5B and 5C differs from the auxiliary portion 42′ of FIGS. 4A, 4B and 4C in that it is configured for forming a loop(s) 67 around the finger(s) 69, said loop(s) 67 anchoring the strap member 14 to the finger(s) and preventing movement of the strap member 14 beyond its appropriate position along the length of the arm 3 when a slider 55 of the zip fastener 39 is moved up along the arm by the person themselves to close the zip fastener.

Preferably, the auxiliary portion 42′ has a finger hole(s) near its distal end, said finger hole(s) configured for anchoring the auxiliary portion 42′ of the strap member to the finger(s) 69 and preventing movement of the strap member 14 beyond its appropriate position along the length of the arm 3 when a slider 55 of the zip fastener 39 is moved up along the arm 3 by the person themselves to close the zip fastener.

FIGS. 2A, 2B and FIGS. 3A, 3B show that the strap member 14 has the waist portion 16 that carries the bladder and a pair of auxiliary portions 41, 42 which extend away from the waist portion 16 in opposite directions along the length of the arm 3. A distal auxiliary portion 42 extends towards the hand, and a proximal auxiliary portion 41 extends towards the shoulder. The waist portion 16 has waist tightening edges 26, 28. The distal auxiliary portion 42 has waist tightening edges 43, 44. The proximal auxiliary portion 41 has waist tightening edges 45, 47. FIGS. 2A, 2B and 3A, 3B illustrates that the waist portion 16 and the auxiliary portions 41, 42 each have a pair of waist tightening edges which form respective segments of the elongated waist tightening edges 30,32 of the strap member 14. It should be understood that the waist portion 16 and the auxiliary portions 41, 42 share their respective waist tightening edges with the elongated waist tightening edges 30,32 of the strap member 14. The waist tightening edges of the waist portion 16 and of both the distal and proximal auxiliary portions 42, 41 of the strap member 14 are attached to the complementary halves 35, 36 of the zip fastener 39, so that the waist portion 16 and the auxiliary portions 42, 41 of the strap member can be closed and opened by the same zip fastener 39.

FIGS. 2A, 2B show that the distal and proximal auxiliary portions 42, 41 of the strap member 14 have central segments 51,52 and peripheral segments 53,54. The central segments 51,53 are located next to the waist portion 16 of the strap member and can be closed and opened by the zip fastener 39 while the peripheral segments 53,54 are spaced away from the waist portion 16 of the strap member and are undividable by the zip fastener 39. The zip fastener 39 extends through the waist portion 16 and the central segments 51,52 of the auxiliary portions 42, 41 and has its slider 55 located such that opening and closing of the strap member 14 is started with one of the two central segments and finished with the other.

It should be understood that each of the auxiliary portions 41, 42 of the strap member 14 shown in FIGS. 2A, 2B is configured for being pulled along the arm in a direction which is opposite to the direction in which the slider 55 of the zip fastener 39 is moved along the arm 3. This prevents movement of the strap member 14 beyond its appropriate position around the arm. Therefore, the proximal auxiliary portion 41 of the strap member 14 is configured for being pulled up along the arm when the slider 55 of the zip fastener 39 is moved down along the arm, and the distal auxiliary portion 42 of the strap member 14 is configured for being pulled down along the arm when the slider 55 of the zip fastener 39 is moved up along the arm. It should be noted that at least one of the slider and the auxiliary portion of the strap member shown in FIGS. 2A and 2B should be manipulated by someone other than the person whose blood pressure is measured.

FIGS. 6A, 6B show that the auxiliary portion 41 of the strap member 14 is continuous with a sleeve 81 of an upper body garment 83. The auxiliary portion 41 has waist tightening edges 45, 47. The waist portion 16 has waist tightening edges 26, 28. FIGS. 6A, 6B illustrate that the waist portion 16 and the auxiliary portion 41 each have a pair of waist tightening edges which they share with the elongated waist tightening edges 30, 32 of the strap member 14. The waist tightening edges of both the waist portion 16 and the auxiliary portion 41 are attached to the complementary halves 35, 36 of the zip fastener 39, so that the waist portion 16 and the auxiliary portion 41 of the strap member can be closed and opened by the same zip fastener 39.

FIGS. 7A, 7B show that the auxiliary portion 41 of the strap member 14 may be formed from only the sleeve of the garment and the zip fastener extending through it.

FIGS. 6A, 6B and FIGS. 7A, 7B show that both auxiliary portions 41,42 of the strap member 14 are either comprised from or continuous with the sleeve 81 of an upper body garment 83. A proximal auxiliary portion 41 is comprised from a portion of the sleeve of the garment which extends from the waist portion 16 up along the arm. A distal auxiliary portion 42 is comprised from a portion of the sleeve 81 of the garment which extends from the waist portion 16 down along the arm. The waist portion 16 has waist tightening edges 26, 28. The distal auxiliary portion 42 has waist tightening edges 43, 44. The proximal auxiliary portion 41 has waist tightening edges 45, 47. FIGS. 6A, 6B and FIGS. 7A, 7B illustrate that the waist tightening edges of the auxiliary portions 41, 42 extend up and down the sleeve 81 of the garment from the waist portion 16 of the strap member to form auxiliary waist tightening edges of the strap member. These auxiliary waist tightening edges are attached to the complementary interlocking halves 35, 36 of the strap member's single zip fastener 39. The strap member's single zip fastener 39 extends through the waist portion 16 of the strap member and continues to extend up and down the sleeve 81 of the garment away from the waist portion 16 of the strap member and has its slider 55 located such that opening and dosing of the strap member is started with one of two auxiliary portions of the strap member and finished with the other.

FIGS. 6A, 6B and FIGS. 7A, 7B illustrate that the waist portion 16 and each of the auxiliary portions have a pair of waist tightening edges which they share with the elongated waist tightening edges 30, 32 of the strap member. The waist tightening edges of the waist portion 16 and of both the distal and proximal auxiliary portions 41, 42 are attached to the complementary halves of the strap member's single zip fastener 39, so that the waist portion and the auxiliary portions of the strap member can be closed and opened by the same zip fastener 39.

FIGS. 6A, 6B and FIGS. 7A, 7B show that the zip fastener 39 extends into a shoulder area 84 of the garment but terminates short of a collar 85 of the garment. FIGS. 8A, 8B show that the zip fastener 39 may extend into the shoulder area 84 of the garment but terminate short of a neck margin 86 of the garment. It should be noted that the zip fastener 39 does not have to extend into the shoulder area of the garment and terminate there as shown in FIGS. 6A through 8B. It is within the scope of the invention for the zip fastener 39 to extend up the sleeve of the garment but terminate short of the shoulder area 84 of the garment.

FIGS. 6A, 6B and FIGS. 7A, 7B show that the distal auxiliary portion 42 of the strap member 14 extends towards the wrist, the waist tightening edges 43,44 of the distal auxiliary portion 42 of the strap member extending down into the forearm area of the sleeve 81 of the garment, so that the sleeve of the garment located down from the waist portion 16 of the strap member can be opened and closed around the arm and forearm by the strap member's single zip fastener.

FIGS. 6A through 9B show that the sleeve 81 has a strap extension 90. The strap extension 90 is configured for anchoring the sleeve to the hand 57, said anchoring of the sleeve 81 to the hand 57 of the arm restricting movement of the strap member 14 up along the arm beyond its appropriate position around the arm when the opposite arm is used to move the slider 55 of the zip fastener 39 up along the arm by the person themselves to close the zip fastener.

It should be noted that the zip fastener 39 may extend down into a forearm portion of a long sleeve 81 of the garment but terminates short of a distal end of the sleeve of the garment. The distal end 87 of the sleeve may have a cuff which extends around the wrist and is configured for anchoring the sleeve to the wrist, said anchoring of the sleeve to the wrist restricting movement of the strap member 14 up along the arm beyond its appropriate position around the arm when the opposite arm is used to move the slider 55 of the zip fastener 39 up along the arm by the person themselves to close the zip fastener.

FIGS. 8A, 8B show that the strap member 14 is continuous with the sleeve 81 of the upper body garment 83. FIGS. 8A, 8B illustrate that the sleeve 81 is one of two sleeves 81, 91 of the upper body garment 83. The strap member 14 extends around the arm which is used for measuring the blood pressure. FIGS. 9A, 9B show that the other sleeve 91 extends around the opposite arm and has a strap extension 93 configured for anchoring the sleeve 91 to the hand of the opposite arm, said anchoring of the other sleeve 91 to the hand of the opposite arm restricting movement of the strap member 14 down along the arm beyond its appropriate position around the arm when the opposite arm is used to move the slider 55 of the zip fastener 39 down along the arm by the person themselves to close the zip fastener.

It should be understood that the other sleeve 91 may have a cuff at its distal end, said cuff being configured for extending around the wrist of the opposite arm and for anchoring the other sleeve 91 to the wrist of the opposite arm, said anchoring of the other sleeve 91 to the wrist of the opposite arm restricting movement of the strap member 14 down along the arm beyond its appropriate position around the arm when the opposite arm is used to move the slider 55 of the zip fastener 39 down along the arm by the person themselves to close the zip fastener.

FIGS. 9A, 9B show that the strap member 14 is continuous with the sleeve 81of the upper body garment 83. The sleeve 81 is one of two sleeves 81, 91 of the upper body garment 83. The strap member 14 extends around the arm which is used for measuring the blood pressure. FIGS. 9A, 9B illustrate that the sleeve 81 has a first strap extension 90 which is configured for anchoring the sleeve 81 to the hand 57 and restricts movement of the strap member 14 up along the arm beyond its appropriate position around the arm when the opposite arm is used to move the slider 55 of the zip fastener 39 up along the arm by the person themselves. FIGS. 10A, 10B illustrate that the other sleeve 91 extends around the opposite arm and has a second strap extension 93 which is configured for anchoring the other sleeve 91 to the hand 96 of the opposite arm and restricts movement of the strap member 14 down along the arm beyond its appropriate position around the arm when the opposite arm is used to move the slider 55 of the zip fastener 39 down along the arm by the person themselves.

It should be noted that the strap extension 93 can be used for anchoring of the other sleeve 91 to the hand 96 of the opposite arm to restrict movement of the strap member 14 down along the arm beyond its appropriate position around the arm when the opposite arm is used to move the slider 55 of the zip fastener 39 down along the arm by the person themselves to close the zip fastener.

According to one embodiment of the invention the strap extension 93 is configured for being grasped by the hand 96 of the opposite arm prior to using the opposite arm and its strap extension holding hand 96 for moving the slider 55 of the zip fastener 39 down along the arm by the person themselves. According to this embodiment of the invention the strap extension 93 is configured for being passed along an outer surface of the hand first, moved under the thumb and across an inner surface of the hand, and finally grasped using fingers prior to using the opposite arm and its strap extension holding hand for moving the slider 55 of the zip fastener 39 down along the arm by the person themselves to close the zip fastener.

FIG. 10A shows that, according to another embodiment of the invention, the strap extension 93 is configured for passing along an outer surface of the hand 96 and forming an anchoring loop 97 around the hand 96, said anchoring loop 97 extending across an inner surface of the hand 96 distal to the base of the thumb 98 but proximal to bases of the fingers 99.

According to yet another embodiment of the invention the strap extension 93 may be configured for passing along an outer surface of the hand 96 and forming an anchoring loop(s) around the finger(s) 99. The strap extension 93 may be provided with a finger hole(s) near its distal end, said finger hole(s) configured for anchoring the strap extension to the finger(s).

FIGS. 10A, 10B and 10C and corresponding transverse cross-sections shown in FIGS. 11A and 11B show that the strap member 14 includes a non-stretchable bridging element 200 which, after closing of the zip fastener 39, is extended over the zip fastener to interconnect regions 211, 212 of the wall of the strap member which adjoin each of the two halves of the zip fastener 39. The non-stretchable bridging element 200 interconnects the regions of the strap member which are located on both sides of the closed zip fastener 39 and prevents the interlocked halves of the zip fastener 39 from separating from each other by reducing the strain applied to the zip fastener 39 when the inflatable bladder 11 is inflated.

FIGS. 11A and 11B show that the non-stretchable bridging element 220 is configured for detachable fastening to the regions 211, 212 of the wall of the strap member 14 located along-sides both halves of the zip fastener 39 by a Velcro brand Hook and Loop fabric fastener. A “hook” side of the fastener may be carried by each of the two regions 211, 212 of the wall of the strap member and a “loop” side by the non-stretchable bridging element 200. Alternatively a “loop” side of the fastener may be carried by each of the two regions of the wall of the strap member and a “hook” side by the non-stretchable bridging element.

FIGS. 12A through 12C illustrate, in transverse cross sections, a modified embodiment in which the strap member 14 includes a stretchable strip 220 which extends along one of the two halves of the zip fastener 39. The non-stretchable bridging element 200 is extended over the stretchable strip 220 and the zip fastener 39 after the zip fastener 39 has been closed to interconnect regions 211, 222 of the wall of the strap member 14 which are spaced away from each other by the stretchable strip 220 and the closed zip fastener 39. The stretchable strip 220 and the non-stretchable bridging element 200 prevent the interlocked halves of the zip fastener from separating from each other by reducing the strain applied to the zip fastener 39 when the inflatable bladder is inflated. The non-stretchable bridging element 200 is configured to prevent the interlocked halves of the zip fastener 39 from separating even when air pressure in the fully inflated bladder has reached about 300 mm mercury, while the stretchable strip 220 is configured to stretch and reduce to a limited extent an initial strain on the zip fastener 39 during an early stage of the inflation when air pressure in the bladder only started to rise. FIG. 12C illustrates such an early stage of inflation.

It should be understood that the stretchable strip 220 of the strap member 14 is configured to stretch only until the strain becomes applied to the non-stretchable bridging element 200 which, when strained, prevents further stretching of the stretchable strip 220.

FIGS. 12A through 12C show that the non-stretchable bridging element 220 is configured for detachable fastening to the regions 211, 222 of the strap member 14 by a Velcro brand Hook and Loop fabric fastener. The region 211 is located along-side one half of the zip fastener 39 and the region 222 is located along-side the stretchable strip 220. The regions 211 and 222 are spaced away from each other by the zip fastener 39 and the stretchable strip 220. A “hook” side of the fastener may be carried by each of the two regions of the strap member 14 and a “loop” side by the non-stretchable bridging element 200. Alternatively a “loop” side of the fastener may be carried by each of the two regions of the strap member 14 and a “hook” side by the non-stretchable bridging element 200.

The non-stretchable bridging element 200 may be permanently attached to one of the two auxiliary portions of the strap member near an end of the zip fastener 39. The non-stretchable bridging element 200 may be permanently attached to the wall of the strap member near either end of the zip fastener 39.

Alternatively the non-stretchable bridging element 200 may be permanently fastened to the region of the strap member 14 located along-side one half of the zip fastener 39 and may be configured for detachable fastening by a Velcro brand Hook and Loop fabric fastener to the region of the strap member located along-side the other half of the zip fastener 39. If the stretchable strip 220 is used than the non-stretchable bridging element 200 may be permanently fastened to the region of the strap member 14 located either along-side one half of the zip fastener 39 or along-side the stretchable strip 220. The regions of permanent and detachable fastening of the non-stretchable bridging element 200 become spaced away from each other by the stretchable strip 220 and the zip fastener 39.

FIG. 11A through 12C show that the waist portion 16 of the strap member 14 has an outer wall 18 and an inner wall 500 and carries the inflatable bladder 11 within a pouch formed between said walls of the waist portion 16 of the strap member 14. Both the pouch and the bladder 11 extend around the arm 3.

Preferably any one of the strap extensions 90,93 should have an adjustable length. The strap extensions may have two parts which are detachably fastened to each other by a Velcro type Hook and Loop fabric fastener. Alternatively, the strap extensions 90,93 may have an adjustable length by being comprised of two parts which are button fastened to each other, one part having a button and the other a plurality of buttonholes spaced away from each other along the length of the other part. In an alternative arrangement, one part of the strap extension has a buttonhole and the other a plurality of buttons spaced away from each other along the length of the other part. Preferably, the hand extension of the distal auxiliary portion 42′ shown in FIGS. 4A through 5B should also be adjustable in length, as has already been described in connection with the strap extensions 90,93.

1. An inflatable cuff comprising an air inflatable bladder and a strap member which forms a sleeve configured to extend around an arm of a person, the sleeve shaped strap member having a waist portion which carries the bladder and has a pair of longitudinally extending edges, wherein the longitudinally extending edges of the waist portion are attached to complimentary interlocking halves of a zip fastener to enable the longitudinally extending edges to be zipped together to reduce the circumference of the waist portion of the sleeve and secure the cuff in position around a person's arm whose blood pressure is to be measured.

2. The inflatable cuff according to claim 1, wherein the bladder extends around the arm under an outer wall of the sleeve shaped strap member, the waist portion being closeable around the arm prior to inflation of the bladder which, after inflation, compresses the arm and stops the flow of blood through the brachial artery of the arm, the waist portion having a circumferential length extending in a direction around the arm and a width extending in a direction along the length of the arm, the waist portion having a pair of waist tightening edges which span the width of the waist portion and which, when the waist portion is open, are spaced away from each other by the circumferential length of the waist portion, the waist tightening edges of the waist portion forming segments of elongated waist tightening edges of the sleeve shaped strap member.

3. The inflatable cuff according to claim 1, wherein the waist tightening edges of the waist portion extend beyond the width of the waist portion to form auxiliary segments of the elongated waist tightening edges of the strap member, the auxiliary segments being attached to the interlocking halves of the zip fastener which also extends beyond the waist portion and which is configured for fastening together all segments of the elongated waist tightening edges of the sleeve shaped strap member.

4. The inflatable cuff according to claim 1, comprising a single zip fastener which fastens together the waist tightening edges.

5. The inflatable cuff according to claim 1, wherein the strap member has the waist portion that carries the bladder and an auxiliary portion which extends along the length of the arm away from the waist portion, both the waist portion and the auxiliary portion having waist tightening edges which form respective segments of the elongated waist tightening edges of the strap member, said waist tightening edges of both the waist portion and the auxiliary portion of the strap member being attached to the complementary halves of the zip fastener, so that the waist portion and the auxiliary portion of the strap member can be closed and opened by the same zip fastener.

6. The inflatable cuff according to claim 1, wherein the strap member has the waist portion that carries the bladder and an auxiliary portion which extends along the length of the arm away from the waist portion, the waist portion and the auxiliary portion each having a pair of waist tightening edges which they share with the elongated waist tightening edges of the strap member and which are attached to the complementary halves of the zip fastener, so that the waist portion and the auxiliary portion of the strap member can be closed and opened by the same zip fastener.

7. The inflatable cuff according to claim 5, wherein the auxiliary portion of the strap member has a central segment and a peripheral segment, the central segment is located next to the waist portion of the strap member and can be closed and opened by the zip fastener while the peripheral segment is spaced away from the waist portion of the strap member and is undividable by the zip fastener, the zip fastener extending through the waist portion of the strap member and the central segment of the auxiliary portion and having a slider located such that its movement opens the waist portion of the strap member before the central segment of the auxiliary portion and closes the central segment of the auxiliary portion before the waist portion of the strap member.

8. The inflatable cuff according to claim 5, wherein the auxiliary portion of the strap member extends up along the arm and is configured for being pulled up along the arm when a slider of the zip fastener is moved down along the arm so that the strap member is not moved beyond its appropriate position along the length of the arm, at least one of the slider and the auxiliary portion of the strap member being manipulated by someone other than the person who's blood pressure is measured.

9. The inflatable cuff according to claim 5, wherein the auxiliary portion of the strap member extends down along the arm and is configured for being pulled down along the arm when a slider of the zip fastener is moved up along the arm so that the strap member is not moved beyond its appropriate position along the length of the arm, at least one of the slider and the auxiliary portion of the strap member being manipulated by someone other than the person who's blood pressure is measured.

10. The inflatable cuff according to claim 5, wherein the auxiliary portion of the strap member extends distally towards the hand and is configured for forming a loop around the hand, said loop anchoring the strap member to the hand and preventing movement of the strap member beyond its appropriate position along the length of the arm when a slider of the zip fastener is moved up along the arm by the person themselves.

11. The inflatable cuff according to claim 10, wherein the auxiliary portion of the strap member extends along an outer surface of the forearm and its anchoring loop extends across the palm.

12. The inflatable cuff according to claim 5, wherein the auxiliary portion of the strap member extends distally towards the hand and is configured for forming a loop around the thumb, said loop anchoring the strap member to the thumb and preventing movement of the strap member beyond its appropriate position along the length of the arm when a slider of the zip fastener is moved up along the arm by the person themselves.

13. The inflatable cuff according to claim 5, wherein the auxiliary portion of the strap member extends distally towards the hand and is configured for forming a loop(s) around the finger(s), said loop(s) anchoring the strap member to the finger(s) and preventing movement of the strap member beyond its appropriate position along the length of the arm when a slider of the zip fastener is moved up along the arm by the person themselves.

14. The inflatable cuff according to claim 5, wherein the auxiliary portion of the strap member extends distally towards the hand and is configured for being grasped by the hand prior to using the opposite arm for moving the slider of the zip fastener up along the arm by the person themselves.

15. The inflatable cuff according to claim 12, wherein the auxiliary portion of the strap member extends along an outer surface of the forearm.

16. The inflatable cuff according to claim 5, wherein the auxiliary portion of the strap member is configured for extending along an outer surface of the forearm and has a finger hole(s) near its distal end, said finger hole(s) configured for anchoring the auxiliary portion of the strap member to the finger(s) and preventing movement of the strap member beyond its appropriate position along the length of the arm when a slider of the zip fastener is moved up along the arm by the person themselves.

17. The inflatable cuff according to claim 10, wherein the auxiliary portion of the strap member has an adjustable length.

18. The inflatable cuff according to 17, wherein the auxiliary portion of the strap member comprises two parts which are detachably fastened to each other by a Velcro brand Hook and Loop fabric fastener to enable the length of the auxiliary portion to be adjusted.

19. The inflatable cuff according to 17, wherein the auxiliary portion of the strap member comprises two parts which are detachably button fastened to each other, one part having a button and the other a plurality of buttonholes spaced away from each other along the length of the other part, to enable the length of the auxiliary portion to be adjusted.

20. The inflatable cuff according to 17, wherein the auxiliary portion of the strap member comprises two parts which are detachably button fastened to each other, one part having a buttonhole and the other a plurality of buttons spaced away from each other along the length of the other part, to enable the length of the auxiliary portion to be adjusted.

21. The inflatable cuff according to claim 1, wherein the strap member has the waist portion that carries the bladder and a pair of auxiliary portions which extend away from the waist portion in opposite directions along the length of the arm, one of the two auxiliary portions being a distal auxiliary portion and extending towards the hand and the other being a proximal auxiliary portion and extending towards the shoulder, the waist portion and the auxiliary portions each having a pair of waist tightening edges which form respective segments of the elongated waist tightening edges of the strap member, said waist tightening edges of the waist portion and of both the distal and proximal auxiliary portions of the strap member being attached to the complementary halves of the zip fastener, so that the waist portion and the auxiliary portions of the strap member can be closed and opened by the same zip fastener.

22. The inflatable cuff according to claim 1, wherein the strap member has the waist portion that carries the bladder and a pair of auxiliary portions which extend away from the waist portion in opposite directions along the length of the arm, one of the two auxiliary portions being a distal auxiliary portion and extending towards the hand and the other being a proximal auxiliary portion and extending towards the shoulder, the waist portion and the auxiliary portions each having a pair of waist tightening edges which they share with the elongated waist tightening edges of the strap member and which are attached to the complementary halves of the zip fastener, so that the waist portion and the auxiliary portions of the strap member can be closed and opened by the same zip fastener.

23. The inflatable cuff according to claim 21, wherein the distal and proximal auxiliary portions of the strap member have central segments and peripheral segments, the central segments are located next to the waist portion of the strap member and can be closed and opened by the zip fastener while the peripheral segments are spaced away from the waist portion of the strap member and are undividable by the zip fastener, the zip fastener extending through the waist portion and the central segments of the auxiliary portions and having a slider located such that opening and closing of the strap member is started with one of the two central segments and finished with the other.

24. The inflatable cuff according to claim 23, wherein each of the auxiliary portions of the strap member is configured for being pulled along the arm in a direction which is opposite to the direction in which the slider of the zip fastener is moved along the arm, so that movement of the strap member beyond its appropriate position around the arm can be prevented, at least one of the slider and the auxiliary portion of the strap member being manipulated by someone other than the person whose blood pressure is measured.

25. The inflatable cuff according to claim 23, wherein the proximal auxiliary portion of the strap member is configured for being pulled up along the arm when the slider of the zip fastener is moved down along the arm so that the strap member is not moved beyond its appropriate position around the arm, at least one of the slider and the proximal auxiliary portion of the strap member being manipulated by someone other than the person whose blood pressure is measured.

26. The inflatable cuff according to claim 23, wherein the distal auxiliary portion of the strap member is configured for being pulled down along the arm when the slider of the zip fastener is moved up along the arm so that the strap member is not moved beyond its appropriate position around the arm, at least one of the slider and the distal auxiliary portion of the strap member being manipulated by someone other than the person whose blood pressure is measured.

27. The inflatable cuff according to claim 1, wherein the strap member has the waist portion that carries the bladder and an auxiliary portion which extends along the length of the arm away from the waist portion and is continuous with a sleeve of an upper body garment, both the waist portion and the auxiliary portion each having a pair of waist tightening edges which they share with the elongated waist tightening edges of the strap member and which are attached to the complementary halves of the zip fastener, so that the waist portion and the auxiliary portion of the strap member can be closed and opened by the same zip fastener.

28. The inflatable cuff according to claim 1, wherein the strap member has the waist portion that carries the bladder and an auxiliary portion which extends along the length of the arm away from the waist portion, both the waist portion and the auxiliary portion of the strap member being continuous with a sleeve of an upper body garment, the waist portion and the auxiliary portion of the strap member having waist tightening edges which they share with the elongated waist tightening edges of the strap member and which are attached to the complementary halves of the zip fastener, so that the waist portion and the auxiliary portion of the strap member can be closed and opened by the same zip fastener.

29. The inflatable cuff according to claim 1, wherein the strap member has the waist portion that carries the bladder and a pair of auxiliary portions which extend away from the waist portion in opposite directions along the length of the arm and are comprised from a sleeve of an upper body garment, a portion of the sleeve of the garment which extends from the waist portion up along the arm comprising a proximal auxiliary portion, and a portion of the sleeve of the garment which extends from the waist portion down along the arm comprising a distal auxiliary portion, each of the auxiliary portions having a pair of waist tightening edges which extend up and down the sleeve of the garment from the waist portion of the strap member to form auxiliary waist tightening edges of the strap member, the auxiliary waist tightening edges being attached to the complementary interlocking halves of the strap member's single zip fastener, the strap member's single zip fastener extending through the waist portion of the strap member and continuing to extend up and down the sleeve of the garment away from the waist portion of the garment and having a slider located such that opening and closing of the strap member is started with one of two auxiliary portions of the strap member and finished with the other.

30. The inflatable cuff according to claim 1, wherein the strap member has the waist portion that carries the bladder and a pair of auxiliary portions which extend away from the waist portion in opposite directions along the length of the arm and which are continuous with a sleeve of an upper body garment, one of two auxiliary portions being a distal auxiliary portion and extending towards the hand and the other being a proximal auxiliary portion and extending towards the shoulder, the waist portion and each of the auxiliary portions having a pair of waist tightening edges which they share with the elongated waist tightening edges of the strap member and which are attached to the complementary halves of the strap member's single zip fastener, so that the waist portion and the auxiliary portions of the strap member can be closed and opened by the same zip fasten.

31. The inflatable cuff according to claim 29, wherein the zip fastener extends up the sleeve of the garment but terminates short of the shoulder area of the garment.

32. The inflatable cuff according to claim 29, wherein the proximal auxiliary portion of the strap member is continuous with sleeve and shoulder areas of the upper body garment, the zip fastener extending into and terminating within the shoulder area of the garment.

33. The inflatable cuff according to claim 29, wherein the proximal auxiliary portion of the strap member is continuous with sleeve and shoulder areas of the upper body garment, the zip fastener extending into the shoulder area of the garment but terminating short of a collar of the garment.

34. The inflatable cuff according to claim 29, wherein the proximal auxiliary portion of the strap member is continuous with sleeve and shoulder areas of the upper body garment, the zip fastener extending into the shoulder area of the garment but terminating short of a neck margin of the garment.

35. The inflatable cuff according to claim 29, wherein the distal auxiliary portion of the strap member extends towards the wrist, the waist tightening edges of the distal auxiliary portion of the strap member extending down into a forearm area of the sleeve of the garment, so that the sleeve of the garment located down from the waist portion of the strap member can be opened and closed around the arm and forearm by the strap member's single zip fastener.

36. The inflatable cuff according to claim 29, wherein the distal auxiliary portion of the strap member is continuous with a long sleeve of the garment, the zip fastener extending down into the forearm portion of the sleeve but terminating short of a distal end of the sleeve of the garment, the distal end of the sleeve having a cuff which extends around the wrist and is configured for anchoring the sleeve to the wrist, said anchoring of the sleeve to the wrist restricting movement of the strap member up along the arm beyond its appropriate position around the arm when the opposite arm is used to move the slider of the zip fastener up along the arm by the person themselves.

37. The inflatable cuff according to claim 29, wherein a portion of the sleeve which is continuous with the distal auxiliary portion of the strap member has a strap extension which is configured for anchoring the sleeve to the hand, said anchoring of the sleeve to the hand of the arm restricting movement of the strap member up along the arm beyond its appropriate position around the arm when the opposite arm is used to move the slider of the zip fastener up along the arm by the person themselves.

38. The inflatable cuff according to claim 29, wherein the strap member is continuous with one of two sleeves of the upper body garment and extends around the arm which is used for measuring the blood pressure, the other sleeve extending around the opposite arm and having a cuff which extends around the wrist and is configured for anchoring the sleeve to the wrist, said anchoring of the other sleeve to the wrist of the opposite arm restricting movement of the strap member down along the arm beyond its appropriate position around the arm when the opposite arm is used to move the slider of the zip fastener down along the arm by the person themselves.

39. The inflatable cuff according to claim 29, wherein the strap member is continuous with one of two sleeves of the upper body garment, the sleeve continuous with the strap member having a first strap extension which is configured for anchoring the sleeve to the hand and restricting movement of the strap member up along the arm when the opposite arm is used to move the slider of the zip fastener up along the arm, the other sleeve extending around the opposite arm and having a second strap extension which is configured for anchoring the other sleeve to the hand of the opposite arm and restricting movement of the strap member down along the arm when the opposite arm is used to move the slider of the zip fastener down along the arm.

40. The inflatable cuff according to claim 29, wherein the strap member is continuous with one of two sleeves of the upper body garment and extends around the arm which is used for measuring the blood pressure, the other sleeve extending around the opposite arm and having a strap extension which is configured for anchoring the other sleeve to the hand of the opposite arm, said anchoring of the other sleeve to the hand of the opposite arm restricting movement of the strap member down along the arm beyond its appropriate position around the arm when the opposite arm is used to move the slider of the zip fastener down along the arm by the person themselves.

41. The inflatable cuff according to claim 40, wherein the strap extension is configured for being grasped by the hand of the opposite arm prior to using the opposite arm and its strap extension holding hand for moving the slider of the zip fastener down along the arm by the person themselves.

42. The inflatable cuff according to claim 40, wherein the strap extension is configured for being passed along an outer surface of the hand first, moved under the thumb and across an inner surface of the hand, and finally grasped using fingers prior to using the opposite arm and its strap extension holding hand for moving the slider of the zip fastener down along the arm by the person themselves.

43. The inflatable cuff according to claim 37, wherein the strap extension is configured for passing along an outer surface of the hand and forming an anchoring loop around the hand, said anchoring loop extending across an inner surface of the hand distal to the base of the thumb but proximal to bases of the fingers.

44. The inflatable cuff according to claim 37, wherein the strap extension is configured for passing along an outer surface of the hand and forming an anchoring loop(s) around the finger (s).

45. The inflatable cuff according to claim 37, wherein the strap extension is configured for passing along an outer surface of the hand and has a finger hole(s) near its distal end, said finger hole(s) configured for anchoring the strap extension to the finger (s).

46. The inflatable cuff according to claim 1, wherein the strap member includes a non-stretchable bridging element which, after closing of the zip fastener, is extended over the zip fastener to interconnect regions of the strap member which adjoin each of the two halves of the zip fastener, the bridging element interconnects the regions of the strap member which are located on both sides of the closed zip fastener and prevents the interlocked halves of the zip fastener from separating from each other by reducing the strain applied to the zip fastener when the inflatable bladder is inflated.

47. The inflatable cuff according to claim 1, wherein the strap member includes a stretchable strip which extends along one of the two halves of the zip fastener and a non-stretchable bridging element which, after closing the zip fastener, is extended over the stretchable strip and the zip fastener to interconnect regions of the strap member which are spaced away from each other by the stretchable strip and the closed zip fastener, the stretchable strip and the non-stretchable bridging element preventing the interlocked halves of the zip fastener from separating from each other by reducing the strain applied to the zip fastener when the inflatable bladder is inflated, the non-stretchable bridging element configured to prevent the interlocked halves of the zip fastener from separating even when air pressure in the fully inflated bladder has reached about 300 mm mercury, while the stretchable strip configured to stretch and reduce to a limited extent an initial strain on the zip fastener during an early stage of the inflation when air pressure in the bladder only started to rise.

48. The inflatable cuff according to claim 47, wherein the stretchable strip of the strap member is configured to stretch only until the strain becomes applied to the non-stretchable bridging element which, when strained, prevents further stretching of the stretchable strip.

49. The inflatable cuff according to claim 46, wherein the non-stretchable bridging element is configured for detachable fastening to the regions of the strap member located along-side both halves of the zip fastener by a Velcro brand Hook and Loop fabric fastener, a “hook” side of the fastener being carried by each of the two regions of the strap member and a “loop” side by the bridging element.

50. The inflatable cuff according to claim 46, wherein the non-stretchable bridging element is configured for detachable fastening to the regions of the strap member located along-side both halves of the zip fastener by a Velcro brand Hook and Loop fabric fastener, a “loop” side of the fastener being carried by each of the two regions of the strap member and a “hook” side by the bridging element.

51. The inflatable cuff according to claim 47, wherein the non-stretchable bridging element is configured for detachable fastening by a Velcro brand Hook and Loop fabric fastener to the regions of the strap member which are spaced away from each other by the zip fastener and the stretchable strip, one of the regions located along-side one half of the zip fastener and the other along-side the stretchable strip, a “hook” side of the fastener being carried by each of the two regions of the strap member and a “loop” side by the bridging element.

52. The inflatable cuff according to claim 47, wherein the non-stretchable bridging element is configured for detachable fastening by a Velcro brand Hook and Loop fabric fastener to the regions of the strap member which are spaced away from each other by the zip fastener and the stretchable strip, one of the regions located along-side one half of the zip fastener and the other along-side the stretchable strip, a “loop” side of the fastener being carried by each of the two regions of the strap member and a “hook” side by the bridging element.

53. The inflatable cuff according to claim 46, wherein the bridging element is permanently attached to the wall of the strap member near either end of the zip fastener.

54. The inflatable cuff according to claim 46, wherein the bridging element is permanently fastened to the region of the strap member located along-side one half of the zip fastener and is configured for detachable fastening by a Velcro brand Hook and Loop fabric fastener to the region of the strap member located along-side the other half of the zip fastener.

55. The inflatable cuff according to claim 47, wherein the bridging element is permanently fastened to the region of the strap member located along-side the stretchable strip and is configured for detachable fastening by a Velcro brand Hook and Loop fabric fastener to the region of the strap member located along-side one half of the zip fastener.

56. The inflatable cuff according to claim 47, wherein the bridging element is permanently fastened to the region of the strap member located along-side one half of the zip fastener and is configured for detachable fastening by a Velcro brand Hook and Loop fabric fastener to the region of the strap member located along-side the stretchable strip.

57. The inflatable cuff according to claim 1, wherein the waist portion of the strap member has inner and outer walls and carries the bladder within a pouch formed between said walls of the waist portion of the strap member, both the pouch and the bladder extending around the arm.

58. The inflatable cuff according to claim 1, wherein the waist portion of the strap member has a pair of longitudinally extending ends, one end coinciding with one of the two waist tightening edges of the waist portion and the other end being spaced away from the other waist tightening edge so that parts of the wall of the waist portion located close to the longitudinally extending ends overlap each other, when the waist portion is closed by the zip fastener.

59. The inflatable cuff according to claim 5 wherein the auxiliary portion(s) of the strap member can be formed from the zip fastener alone.

60. The inflatable cuff according to claim 5, wherein the auxiliary portion(s) of the strap member can be formed from only the sleeve of the garment and the zip fastener which extends through the sleeve.

61. The inflatable cuff according to claim 1 wherein the waist portion of the strap member is configured such that, when the zip fastener is open, the edges of the sleeve are separated from each other to enable a person to insert their arm into the sleeve from one end of the sleeve shaped strap member.

62. The inflatable cuff according to claim 37, wherein any of the strap extension(s) have an adjustable length.
